(19) United States
(12) Patent Application Publication
(10) Pub. No.: US 2005/0175616 A1
(43) Pub. Date: Aug. 11, 2005

(54) PCDGF receptor antibodies and methods of use thereof

(76) Inventors: Peter Kiener, Potomac, MD (US); Solomon Langermann, Baltimore, MD (US); Michael Kinch, Laytonsville, MD (US); Elizabeth Bruckheimer, Rockville, MD (US)

Correspondence Address: JOHNATHAN KLEIN-EVANS, ONE MEDIMMUNE WAY, GAITHERSBURG, MD, 20878, US

(21) Appl. No.: 10/854,326

(22) Filed: May 26, 2004

Related U.S. Application Data
(60) Provisional application No. 60/474,493, filed on May 30, 2003.
(60) Provisional application No. 60/478,908, filed on Jun. 16, 2003.
(60) Provisional application No. 60/487,411, filed on Jul. 15, 2003.

(51) Int. Cl. A61K 39/395; C07K 16/30

(52) U.S. Cl. 424/155.1; 530/388.8

(57) ABSTRACT

The present invention relates to the use of antitumor compositions capable of inhibiting PCDGF biological activity for the treatment of cancers. In particular the present invention relates to the use of PCDGF antagonist, e.g., antibodies that prevent PCDGF from binding its receptor, Rse (also referred to as “Sky” and “Tyro3”).

This application claims the benefit under 35 U.S.C § 119(e) of U.S. Provisional Application No. 60/474,493, filed on May 30, 2003; U.S. Provisional Application No. 60/478,908, filed on Jun. 16, 2003 and U.S. Provisional Application No. 60/487,411, filed on Jul. 15, 2003, each of which is incorporated herein by reference in its entirety.

### BACKGROUND OF THE INVENTION

Cancer Cell Signaling

Cancer is a disease of aberrant signal transduction. Aberrant cell signaling overrides anchorage-dependent constraints on cell growth and survival (Rhim, et al., Critical Reviews in Oncogenesis 8:305, 1997; Patarca, Critical Reviews in Oncogenesis 7:343, 1996; Malik, et al., Biochimica et Biophysica Acta 1287:73, 1996; Cancer, et al., Breast Cancer Res Treat 35:105, 1995). Tyrosine kinase activity is induced by ECM anchorage and indeed, the expression or function of tyrosine kinases is usually increased in malignant cells (Rhim, et al., Critical Reviews in Oncogenesis 8:305,1997; Cance, et al., Breast Cancer Res Treat 35:105, 1995; Hunter, Cell 88:333, 1997). Based on evidence that tyrosine kinase activity is necessary for malignant cell growth, tyrosine kinases have been targeted with new therapeutics (Levitzki, et al., Science 267:1782, 1995; Kondapaka, et al., Molecular & Cellular Endocrinology 117:53, 1996; Fry, et al., Current Opinion in BioTechnology 6: 662, 1995). Unfortunately, obstacles associated with specific targeting to tumor cells often limit the application of these drugs. In particular, tyrosine kinase activity is often vital for the function and survival of benign tissues (Levitzki, et al., Science 267:1782, 1995).

PC-Cell-Derived Growth Factor (“PCDGF”)

PC-cell-derived growth factor (“PCDGF”) is an 88-kDa glycoprotein autocrine growth factor expressed in a tightly regulated fashion in normal cells but overexpressed and unregulated in tumorigenic cells. See, e.g., U.S. Publication No. US20020183270 and U.S. Pat. No. 6,309,826. Inhibition of PCDGF expression or activity inhibits the growth of tumorigenic cells. Id. PCDGF is also known in the art as “GP88.” Id.

Amino-acid and cDNA sequencing indicated that PCDGF is identical to the precursor of epithelins/granulins first purified as 6 kDa double cysteine-rich polypeptides from rat kidney or human granulocyte extracts. See, Serrero et al., PNAS 2001 98(1):142-7. The sequence of mouse and human GP88 (PCDGF) is publicly available. See, e.g., U.S. Publication No. US20020183270 and U.S. Pat. No. 6,309,826. The granulin/epithelin precursor was previously thought to be inactive (see U.S. Pat. No. 5,416,192), however, Serrero et al. demonstrated that PCDGF is a highly active, tumorigenic protein associated with a variety of tumor cell types. See, e.g., U.S. Publication No. US20020183270 and U.S. Pat. No. 6,309,826. The degree of overexpression of PCDGF positively correlates with the degree of tumorigenicity of cells. Id.

PCDGF is a growth modulator for a variety of cell lines, including fibroblasts, PC cells, and mammary epithelial cells. Comparison of the expression of PCDGF in the highly tumorigenic PC cells and in parent 1246 cells demonstrated that PCDGF expression was very low in the non-tumorigenic cells and was overexpressed in the highly tumorigenic cells. See, e.g., Zhang and Serrero PNAS 1998 24;95(24):14202-7. It has also been observed that PCDGF is overexpressed in ovarian tumor samples. For example, PCDGF was expressed only in the invasive ovarian cancer libraries and was absent in the LMP (low malignant potential) libraries. Jones et al., Clin Cancer Res 2003 (1):44-51.

PCDGF antagonists (e.g., anti-PCDGF antibodies and PCDGF antisense nucleic acids) inhibit or interfere with the activity of PCDGF and with the growth of tumorigenic cells. See, Zhang and G. Serrero, 1998, PNAS 95, no. 24:14202; Lu and Serrero, 2000, PNAS 97, no. 8:3993; and Jones et al., Clin Cancer Res 2003 (1):44-51. In both teratoma-derived cells and breast cancer cells, PCDGF activity was inhibited by treating the cells with an anti-PCDGF neutralizing antibody or by transfecting the cells with an antisense PCDGF cDNA. Treatment of cells with PCDGF antagonists in teratoma cells or breast carcinoma cells completely inhibited cell proliferation and tumorigenesis in vivo. Id.

Rse (also Known as “Tyro3” and “Sky”) Tyrosine Kinase

Rse (also known as “Tyro3” and “Sky”) is a member of the Ax1/Sky/Mer receptor tyrosine kinase family. Funakoshi et al., J Neurosci Res 2002 68(2): 150-60. Mark et al. described the human and murine complementary DNA sequences of the receptor tyrosine kinase Rse that is preferentially expressed in the adult brain (Mark et al., J. Biol. Chem. 269: 10720 [1994]). The extracellular domain of Rse receptor is composed of two immunoglobulin-like (Ig-L) repeats followed by two fibronectin type III repeats. Complementary DNA sequences encoding proteins identical to human (Ohashi et al., Oncogene 9: 699 [1994]) and murine Rse (Lai et al., Oncogene 9: 2567 [1994]) have been reported independently, and termed “Sky” and “Tyro3,” respectively. See also Fujmimoto and Yamamoto, Oncogene 9: 693 (1994).

The expression of Rse in various tissues has been investigated. Lai et al., supra, found that, in the adult brain, Rse mRNA is localized in neurons of the neocortex, cerebellum and hippocampus. Schulz et al. similarly found that Rse is expressed at high levels in the cerebral cortex, the lateral septum, the hippocampus, the olfactory bulb and in the cerebellum. The highest levels of Rse expression in the brain were found to be associated with neurons. (Schulz et al. Molec. Brain Res. 28: 273-280 [1995]). In the central nervous system (CNS) of mice, the expression of Rse is detected at highest levels during late embryonic stages and post birth, coincident with the establishment and maintenance of synaptic circuitry in cortical and hippocampal neurons (Lai et al, supra and Schneider et al, Cell 54: 787-793 [1988]). This process is believed to be regulated locally, by cells that are in direct contact or positioned close to one another. By Northern blot analysis, Mark et al., supra, found that high levels of Rse mRNA were present in samples of RNA from the brain and kidney. Dai et al., supra found that Rse was highly expressed in human ovary and testes. The expression of Rse in various human cell lines was also analyzed by Mark et al., supra. Little, or no, Rse mRNA was detected by Northern blotting of mRNA samples from the monocyte cell line THP-1 or the lymphoblast-like RAJI cells. However, the Rse transcript was detected in a number of hematopoietic cell lines, including cells of the myeloid (i.e., myelogenous leukemia line K562 and myelomonocytic U937 cells) and the megakaryocytic leukemia lines DAMI and CMK11-5, as well as the human breast carcinoma cell line MCF-7. In the cell lines examined, a high level of expression was also observed in Hep 3B cells, a human hepatocarcinoma cell line.

In addition, it has been reported that sky mRNA is significantly more abundant in mammary tumors isolated from transgenic mice than in hyperplastic mammary glands and mammary glands of virgin females. Taylor et al., J Biol Chem 1995 270(12):6872-80. Further, Sky mRNA levels are up-regulated in human mammary epithelial cells but not in fibroblasts after transformation with SV40 large T antigen and v-Ha-ras. Moreover, RatB1a fibroblasts overexpressing murine Sky exhibit a transformed morphology, grow as colonies in soft agar, and form tumors when injected into nude mice.

The amino acid sequence of the human Sky and murine Sky homologs are 90% identical, although the human protein contains an additional 10 amino acid residues in the putative signal peptide sequence. Mark et al., (1994) J. Biol Chem. 269, 10720-10728. Rse is structurally related to AxI (also known as Ufo or Ark) and shares 43% overall amino acid sequence identity with this tyrosine kinase receptor. See, O'Bryan et al, Mol. Cell. Biol. 11: 5016 (1991), Janssen et al, Oncogene 6: 2113 (1991), Rescigno et al., Oncogene 5: 1908 (1991) and Bellosta et al., Mol Cell Biol. 15(2): 614 (1995).

The polynucleotide and amino acid sequence of Rse and splice variants thereof are well known in the art. See, e.g., Genbank Accession Nos. BC051756 and AAH51756, respectively. See also, Genseq Accession Nos. AAB27663 (International Publication WO00/53757); ABG79688 (US2002086384); AAR60548; and ARR60660 (Extracellular domain). All of the above mentioned applications are incorporated by reference herein. For example, see full-length Rse polynucleotide and polypeptide sequences as SEQ ID NOS:1 and 2 respectively.

Rse Ligands

It has been previously reported that human Gas6 (growth arrest-specific gene 6) can act as a ligand for both human Rse (Godowski et al., Cell. 1995 Aug. 11;82(3):355-8 and Mark et al., J Biol Chem. 1996 Apr. 19;271(16):9785-9) and human Ax1 (Varnum et al., Nature. 1995 Feb. 16;373(6515):623-6). Gas6 belongs to a set of murine genes that are highly expressed during serum starvation in NIH 3T3 cells (Schneider et al., Cell 54: 787-793 [1988]). These genes were designated growth arrest-specific genes, since their expression is negatively regulated during growth induction. The human homolog of murine gas6 was also cloned and sequenced by Manfioletti et al. (Molec. Cell Biol. 13(8): 4976-4985 (1993)). They concluded that Gas6 is a vitamin K-dependent protein and speculated that it may play a role in the regulation of a protease cascade relevant in growth regulation. Gas6 is expressed in a variety of tissues including the brain. See also Colombo et al. Genome 2: 130-134 (1992) and Ferrero et al., J. Cellular Physiol, 158: 263-269 (1994) concerning Gas6. All of these references are incorporated by reference herein in their entirety.

Until this report herein by the inventors, it was not known that PCDGF was a ligand of Rse. Gas6 and PCDGF are not homologous peptides at the amino acid or polynucleotide level.

Receptor Tyrosine Kinases Ax1 and Mer

Receptor tyrosine kinases Ax1 and Mer are members of the same receptor tyrosine kinase family as Tyro3 and have been described previously. See, e.g., McCloskey et al., (1997) J. Biol. Chem., 272 (37) 23285-23291; O'Bryan et al. (1991) Mol. Cell. Biol. 11, 5016-5031; Chen et al., (1997) Oncogene 14(17):2033-9; Crosier et al., (1997) Pathology. 29(2):131-5; Collett et al., (2003) Circ Res. 92(10):1123-9; U.S. Pat. Nos. 5,468,634 and 5,585,269. These references are incorporated by reference herein in their entirety.

Cancer Therapy

There is a significant need for alternative cancer treatments, particularly for treatment of cancer that has proved refractory to standard cancer treatments, such as surgery, radiation therapy, chemotherapy, and hormonal therapy. Further, it is uncommon for cancer to be treated by only one method. Thus, there is a need for development of new therapeutic agents for the treatment of cancer and new, more effective, therapy combinations for the treatment of cancer.

### BRIEF SUMMARY OF THE INVENTION

The present invention is based, in part, on the inventors' discovery that a receptor for PCDGF is Rse, also referred to as “Sky” and “Tyro3” in the art.

PCDGF is a highly tumorigenic autocrine growth factor and causative agent for a wide variety of tumors. See, e.g., U.S. Pat. No. 6,309,826, which is incorporated by reference herein in its entirety. Overexpression of PCDGF leads to uncontrolled cell growth and increased tumorigenesis. Id. The degree of PCDGF overexpression directly correlates with the degree of cellular tumorigenicity.

As one embodiment, the present invention provides antitumor compositions capable of preventing or inhibiting the binding of PCDGF to the surface of a cell expressing Rse.

The invention further provides antitumor compositions capable of preventing or inhibiting Rse and PCDGF from binding their respective binding partners.

The invention further provides a method of treating cancers that express Rse, including but not limited to ovarian, breast, liver, prostate, kidney, brain, blood cell cancers, hematopoietic cell cancers, other hyperproliferative diseases, and precancerous conditions (e.g., PIN) with antitumor compositions disclosed herein.

The invention further provides a method of treating cancers of cells that do not express Rse with antitumor compositions disclosed herein. More specifically, the invention provides a method of regulating cancerous cells that do not express Rse, but are regulated by the biological activity (e.g., signaling cascade) resulting from Rse activation on other cells (i.e., indirectly activated by downstream effectors).

Antitumor compositions include, for example, antagonists (e.g., antibodies) that prevent or inhibit PCDGF from binding to Rse; prevent or inhibit Rse and PCDGF from binding other binding partners; antagonists (e.g., antibodies) that inhibit PCDGF biological activity; antagonists (e.g., antibodies) that inhibit Rse biological activity; and antagonists (e.g., antibodies) that inhibit PCDGF biological activity.

In an alternative embodiment, antitumor compositions include agonistic molecules (e.g., antibodies) that, although they initially activate Rse, ultimately inhibit Rse or PCDGF expression in a cell by causing Rse degradation.

Antagonists of the invention may, for example, interfere or inhibit binding of PCDGF to the PCDGF receptor by binding PCDGF or by binding Rse or binding both PCDGF and Rse. Such antibodies will be capable of inhibiting the biological activity of PCDGF and/or Rse, including, but not limited to, tumor cell proliferation induced by PCDGF.

Anti-PCDGF antibodies and/or antibody fragments can be made using one of the methods well known in the art, for example, by immunizing an animal with PCDGF polypeptides or fragments or variants thereof. The resulting anti-PCDGF or anti-Rse antibodies or antibody fragments thereof can be used to reduce the proliferation of tumor cells in vitro and in vivo.

The invention provides, in one embodiment, antitumor compositions comprising an antibody or antibody fragment capable of binding to the surface of a cell expressing the PCDGF receptor and interfering with the binding of PCDGF to Rse.

In a preferred embodiment, it is specifically contemplated that a PCDGF antagonist of the invention can be used in combination with an agonist or antagonist of the EphA2 and/or EphA4 receptors for the treatment of cancer. Examples of EphA2 and EphA4 agonist and antagonist molecules are described in U.S. patent application Ser. No. 09/640,935 filed Aug. 17, 2000; Ser. No. 09/952,560 filed Sep. 12, 2001; Ser. No. 09/640,952 filed Aug. 17, 2000; Ser. No. 10/436,783 filed May 12, 2003; Ser. No. 10/436,782 filed May 12, 2003; 60/503,356 filed Sep. 16, 2003 and 60/476,909 filed Jun. 6, 2003, each of which is incorporated by reference herein in its entirety.

Another preferred embodiment of the invention is a method of treating or preventing ER (estrogen receptor)-positive and ER-negative breast cancer, as well as, other cancers (e.g., prostate, ovarian, lung, liver, brain, hematopoietic cell cancers, blood cell cancers) by administering to a patient a therapeutically effective amount of PCDGF or Rse antagonists. In a specific embodiment, said antagonists are antibodies which interfere with PCDGF binding to Rse. In another specific embodiment, said antagonists are antibodies which interfere with PCDGF binding to Rse, but does not substantially prevent Gas6 binding to Rse.

In a specific preferred embodiment, antagonists of the invention are antibodies that prevent or interfere with PCDGF binding to Rse, but do not substantially prevent Gas6 binding to Rse. For example, antagonists of the invention prevent or interfere with PCDGF binding to Rse, but preferably only inhibit Gas6 binding by less than 1%, or by less than 5%, or by less than 10%, or by less than 20%, or by less than 30%, or by less than 40%, or by less than 50%, or by less than 60%, or by less than 70%, or by less than 80% when compared to binding in the absence of the antagonist.

Yet another preferred embodiment of the invention is a method of treating cancer drug insensitivity by administering to a patient a therapeutically effective amount of PCDGF or Rse antagonists. In one specific embodiment, said antagonists are antibodies which interfere with PCDGF binding to Rse. In another specific embodiment, said antagonists are antibodies which prevent or interfere with PCDGF binding to Rse, but does not substantially prevent Gas6 binding to Rse. For example, antagonists of the invention prevent or interfere with PCDGF binding to Rse, but preferably only inhibit Gas6 binding by less than 1%, or by less than 5%, or by less than 10%, or by less than 20%, or by less than 30%, or by less than 40%, or by less than 50%, or by less than 60%, or by less than 70%, or by less than 80% when compared to binding in the absence of the antagonist.

In another preferred embodiment, PCDGF receptor antagonists and PCDGF antagonists of the invention inhibit the expression and/or activity of Rse receptor at least 5-fold, or at least 4-fold, or a least 3-fold, or at least 2-fold, or at least 50%, or at least 25%, when compared to Rse activity or levels of Rse expression in the presence of Gas6, but in the absence of said antagonist.

Another preferred embodiment of the invention is a method of treating patients that are non-responsive to currently available therapies (e.g., cancer therapies) such as chemotherapy, radiation therapy, surgery, hormonal therapy and/or biological therapy/immunotherapy by administering to a patient a therapeutically effective amount of PCDGF or Rse antagonists.

Additional embodiments and advantages of the present invention will be set forth in part in the description that follows, and in part will be obvious from the description, or may be learned through the practice of the invention. The objects and advantages of the invention will be attained by means of the instrumentalities and combinations particularly pointed out in the appended claims.

### DEFINITIONS

The term “antagonist of PCDGF” and “antagonist of Rse” (or the PCDGF receptor) as used herein refers to any molecule, including, without limitation, antibodies, peptides, small molecules, antisense molecules, inhibitory RNA, and ribozymes, which are capable of inhibiting or preventing 1) the binding of PCDGF with Rse; or 2) the binding of Rse or PCDGF with other binding partners; or 3) the biological activity of Rse or PCDGF; or 4) the expression of Rse or PCDGF by a cell.

The term “PCDGF antibody of the invention” or “PCDGF receptor antibody of the invention” or “Rse antibody of the invention” as used herein refers to antibodies or fragments thereof that immunospecifically bind to a PCDGF polypeptide or Rse, or a fragment thereof.

The terms “PCDGF receptor”and “Rse” as used herein are used interchangeably.

The term “antibodies or fragments thereof that immunospecifically bind to PCDGF” or “antibodies or fragments thereof that immunospecifically bind to Rse (or the PCDGF receptor)” as used herein refers to antibodies or fragments thereof that specifically bind to a PCDGF polypeptide or Rse, or a fragment thereof, and do not specifically bind to other polypeptides. Preferably, antibodies or fragments that immunospecifically bind to PCDGF or Rse, or fragments thereof, do not cross-react with other antigens. Antibodies or fragments that immunospecifically bind to a PCDGF or Rse, or a fragment thereof can be identified, for example, by immunoassays or other techniques known to those of skill in the art. Antibodies of the invention include, but are not limited to, synthetic antibodies, monoclonal antibodies, recombinantly produced antibodies, intrabodies, multispecific antibodies, human antibodies, humanized antibodies, chimeric antibodies, synthetic antibodies, single-chain Fvs (scFv), Fab fragments, F(ab′) fragments, disulfide-linked Fvs (sdFv), and anti-idiotypic (anti-Id) antibodies, and epitope-binding fragments of any of the above. In particular, antibodies of the present invention include immunoglobulin molecules and immunologically active portions of immunoglobulin molecules, i.e., molecules that contain an antigen binding site that immunospecifically binds to an PCDGF antigen (e.g., one or more complementarity determining regions (CDRs) of an anti-PCDGF antibody). Preferably antagonistic antibodies or fragments that immunospecifically bind to PCDGF or Rse, or a fragment thereof, preferentially antagonize PCDGF and do not significantly antagonize other activities.

The term “derivative” or “variant” as used herein refers to a polypeptide that comprises an amino acid sequence of PCDGF or Rse, or a fragment thereof, an antibody that immunospecifically binds to PCDGF or Rse; or an antibody fragment that immunospecifically binds to PCDGF or Rse, that has been altered by the introduction of amino acid residue substitutions, deletions or additions. The term “derivative” or “variant” as used herein also refers to PCDGF or Rse, or a fragment thereof; an antibody that immunospecifically binds to PCDGF or Rse; or an antibody fragment that immunospecifically binds to PCDGF or Rse which has been modified, i.e., by the covalent attachment of any type of molecule to the polypeptide. For example, but not by way of limitation, a PCDGF or Rse polypeptide, a fragment of a PCDGF or Rse polypeptide, an antibody, or antibody fragment may be modified, e.g., by glycosylation, acetylation, pegylation, phosphorylation, amidation, derivatization by known protecting/blocking groups, proteolytic cleavage, linkage to a cellular ligand or other protein, etc. A derivative or variant of a PCDGF or Rse polypeptide, or a fragment thereof, an antibody, or antibody fragment may be modified by chemical modifications using techniques known to those of skill in the art, including, but not limited to, specific chemical cleavage, acetylation, formylation, metabolic synthesis of tunicamycin, etc. Further, a derivative or variant of a PCDGF or Rse polypeptide, or a fragment thereof, an antibody, or antibody fragment may contain one or more non-classical amino acids. In one embodiment, a polypeptide derivative/variant possesses a similar or identical function as a PCDGF polypeptide, a fragment of a PCDGF polypeptide, an antibody, or antibody fragment described herein. In another embodiment, a derivative of a PCDGF or Rse polypeptide, or a fragment thereof, an antibody, or antibody fragment has an altered activity when compared to an unaltered polypeptide. For example, a derivative antibody or fragment thereof can bind to its epitope more tightly or be more resistant to proteolysis.

The term “epitope” as used herein refers to portions of a PCDGF or Rse polypeptide, or a fragment thereof having antigenic or immunogenic activity in an animal, preferably in a mammal, and most preferably in a human. An epitope having immunogenic activity is a portion of a PCDGF or Rse polypeptide, or a fragment thereof, that elicits an antibody response in an animal. An epitope having antigenic activity is a portion of a PCDGF or Rse polypeptide, or a fragment thereof, to which an antibody immunospecifically binds as determined by any method well known in the art, for example, by immunoassays. Antigenic epitopes need not necessarily be immunogenic.

The “fragments” described herein include a peptide or polypeptide comprising an amino acid sequence of at least 5 contiguous amino acid residues, at least 10 contiguous amino acid residues, at least 15 contiguous amino acid residues, at least 20 contiguous amino acid residues, at least 25 contiguous amino acid residues, at least 40 contiguous amino acid residues, at least 50 contiguous amino acid residues, at least 60 contiguous amino residues, at least 70 contiguous amino acid residues, at least contiguous 80 amino acid residues, at least contiguous 90 amino acid residues, at least contiguous 100 amino acid residues, at least contiguous 125 amino acid residues, at least 150 contiguous amino acid residues, at least contiguous 175 amino acid residues, at least contiguous 200 amino acid residues, or at least contiguous 250 amino acid residues of the amino acid sequence of a PCDGF or Rse polypeptide, or a fragment thereof, or an antibody that immunospecifically binds to a PCDGF or Rse polypeptide, or a fragment thereof. Preferably, antibody fragments are epitope-binding fragments.

As used herein, the term “humanized antibody” refers to forms of non-human (e.g., murine) antibodies or chimeric antibodies that contain minimal sequence derived from non-human immunoglobulin. For the most part, humanized antibodies are human immunoglobulins (recipient antibody) in which hypervariable region residues of the recipient are replaced by hypervariable region residues from a non-human species (donor antibody) such as mouse, rat, rabbit or non-human primate having the desired specificity, affinity, and capacity. In some instances, Framework Region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable regions correspond to those of a non-human immunoglobulin and all or substantially all of the FRs are those of a human immunoglobulin sequence. The humanized antibody optionally also will comprise at least a portion of an immunoglobulin constant region (Fc), typically that of a human immunoglobulin. For further details, see Jones et al., 1986, Nature 321:522-525; Reichmann et al., 1988, Nature 332:323-329; Presta, 1992, Curr. Op. Struct. Biol. 2:593-596; and Queen et al., U.S. Pat. No. 5,585,089.

As used herein, the term “hypervariable region” refers to the amino acid residues of an antibody that are responsible for antigen binding. The hypervariable region comprises amino acid residues from a “Complementarity Determining Region” or “CDR” (i.e. residues 24-34 (L1), 50-56 (L2) and 89-97 (L3) in the light chain variable domain and 31-35 (H1), 50-65 (H2) and 95-102 (H3) in the heavy chain variable domain; Kabat et al., Sequences of Proteins of Immunological Interest, 5th Ed. Public Health Service, National Institutes of Health, Bethesda, Md. (1991)) and/or those residues from a “hypervariable loop” (i.e. residues 26-32 (L1), 50-52 (L2) and 91-96 (L3) in the light chain variable domain and 26-32 (H1), 53-55 (H2) and 96-101 (H3) in the heavy chain variable domain; Chothia and Lesk, 1987, J. Mol. Biol. 196:901-917). “Framework Region” or “FR” residues are those variable domain residues other than the hypervariable region residues as herein defined.

As used herein, the phrase “non-responsive/refractory” is used to describe patients treated with currently available therapies (e.g., cancer therapies) such as chemotherapy, radiation therapy, surgery, hormonal therapy and/or biological therapy/immunotherapy wherein the therapy is not clinically adequate to treat the patients such that these patients need additional effective therapy, e.g., remain unsusceptible to therapy. The phrase can also describe patients who respond to therapy yet suffer from side effects, relapse, develop resistance, etc. In various embodiments, “non-responsive/refractory” means that at least some significant portion of the cancer cells are not killed or their cell division arrested. The determination of whether the cancer cells are “non-responsive/refractory” can be made either in vivo or in vitro by any method known in the art for assaying the effectiveness of treatment on cancer cells, using the art-accepted meanings of “refractory” in such a context. In various embodiments, a cancer is “non-responsive/refractory” where the number of cancer cells has not been significantly reduced, or has increased during the treatment.

As used herein, the terms “single-chain Fv” or “scFv” refer to antibody fragments comprise the VH and VL domains of antibody, wherein these domains are present in a single polypeptide chain. Generally, the Fv polypeptide further comprises a polypeptide linker between the VH and VL domains which enables the scFv to form the desired structure for antigen binding. For a review of scFv see Pluckthun in The Pharmacology of Monoclonal Antibodies, vol. 113, Rosenburg and Moore eds. Springer-Verlag, New York, pp. 269-315 (1994).

“Stringent hybridization conditions”—In one, non limiting example stringent hybridization conditions are hybridization at 6×sodium chloride/sodium citrate (SSC) at about 45° C., followed by one or more washes in 0.1×SSC, 0.2% SDS at about 68° C. In a preferred, non-limiting example stringent hybridization conditions are hybridization in 6×SSC at about 45° C., followed by one or more washes in 0.2×SSC, 0.1% SDS at 50-65° C. (i.e., one or more washes at 50° C., 55° C., 60° C. or 65° C.). It is understood that the nucleic acids of the invention do not include nucleic acid molecules that hybridize under these conditions solely to a nucleotide sequence consisting of only A or T nucleotides.

### DETAILED DESCRIPTION OF THE INVENTION

The present invention is based, in part, on the inventors' discovery that a receptor for PCDGF is Rse, also referred to as “Sky” and “Tyro3” in the art.

The present invention provides antitumor compositions capable of inhibiting PCDGF biological activity. In addition, based on the fact that the Rse binds PCDGF, the present invention further provides antitumor compositions capable of inhibiting Rse biological activity (e.g., signaling by PCDGF).

As one embodiment, the present invention provides antitumor compositions capable of preventing or inhibiting the binding of PCDGF to the surface of a cell expressing the PCDGF receptor, Rse.

The invention further provides antitumor compositions capable of preventing or inhibiting Rse and PCDGF from binding their respective binding partners.

The invention further provides a method of treating cancers (e.g., ovarian, breast, liver, prostate, kidney, testes, brain, cancers of blood cells and hematopoietic cells) other hyperproliferative diseases, and precancerous conditions (e.g., PIN) with antitumor compositions of the invention. See, Hyperproliferative Diseases infra

In a specific preferred embodiment, PCDGF receptor antagonists of the invention prevent or inhibit the binding of PCDGF to Rse, but do not prevent and/or substantially inhibit the binding of Gas6 to Rse. Preferably, the PCDGF receptor antagonists inhibit Gas6 binding by less than 1%, or by less than 5%, or by less than 10%, or by less than 20%, or by less than 30%, or by less than 40%, or by less than 50%, or by less than 60%, or by less than 70%, or by less than 80% when compared to binding in the absence of the Rse antagonist.

Another preferred embodiment of the invention is a method of treating or preventing ER (estrogen receptor)-positive and ER-negative breast cancer, as well as, other cancers (e.g., prostate, ovarian, lung, liver, brain, hematopoietic cell cancers, blood cell cancers) by administering to a patient a therapeutically effective amount of PCDGF or Rse antagonists. In a specific embodiment, said antagonists are antibodies which interfere with PCDGF binding to Rse. In another specific embodiment, said antagonists are antibodies which interfere PCDGF binding to Rse, but does not substantially prevent Gas6 binding to Rse. For example, antagonists of the invention prevent or interfere with PCDGF binding to Rse, but preferably only inhibit Gas6 binding by less than 1%, or by less than 5%, or by less than 10%, or by less than 20%, or by less than 30%, or by less than 40%, or by less than 50%, or by less than 60%, or by less than 70%, or by less than 80% when compared to binding in the absence of the antagonist.

Yet another preferred embodiment of the invention is a method of drug treating cancer drug insensitivity by administering to a patient a therapeutically effective amount of PCDGF or Rse antagonists. In a specific embodiment, said antagonists are antibodies which interfere with PCDGF binding to Rse. In another specific embodiment, said antagonists are antibodies which interfere PCDGF binding to Rse, but does not substantially prevent Gas6 binding to Rse. For example, antagonists of the invention prevent or interfere with PCDGF binding to Rse, but preferably only inhibit Gas6 binding by less than 1%, or by less than 5%, or by less than 10%, or by less than 20%, or by less than 30%, or by less than 40%, or by less than 50%, or by less than 60%, or by less than 70%, or by less than 80% when compared to binding in the absence of the antagonist.

Another preferred embodiment of the invention is a method of treating patients that are non-responsive to currently available therapies (e.g., cancer therapies) such as chemotherapy, radiation therapy, surgery, hormonal therapy and/or biological therapy/immunotherapy by administering to a patient a therapeutically effective amount of PCDGF or Rse antagonists.

Antitumor compositions of the present invention include PCDGF antagonists and Rse antagonists.

In one preferred embodiment, PCDGF antagonists of the invention may bind the active site of PCDGF (e.g., the PCDGF receptor binding site) and prevent PCDGF from binding to its receptor, Rse. Alternatively, PCDGF antagonists of the invention may bind to a site on PCDGF other than the active site, alter the conformation of the active site, and thus render PCDGF incapable of binding to its receptor.

In another preferred embodiment, PCDGF receptor antagonists of the invention prevent or inhibit the binding of PCDGF to Rse. In a specific embodiment, PCDGF receptor antagonists bind the extracellular domain of Rse polypeptide or a fragment thereof.

In another preferred embodiment, PCDGF receptor antagonists and PCDGF receptor antagonists of the invention interfere or inhibit the interaction of PCDGF and Rse with their respective binding partners.

In another preferred embodiment, PCDGF receptor antagonists and PCDGF antagonists of the invention inhibit the expression and/or activity of the Rse receptor by, for example, by interfering with the binding of PCDGF to Rse itself and/or a coreceptor and/or another tyrosine kinase receptor (e.g., Ax1 and Mer) and/or Gas6.

In another preferred embodiment, PCDGF receptor antagonists and PCDGF antagonists of the invention interfere with the binding of Gas6 to Ax1 and/or Mer, and/or Rse.

In addition, PCDGF receptor antagonists and PCDGF antagonists of the invention preferably alter the intensity, duration, or specific downstream signaling pathways mediated by Gas6-Ax1 and/or Gas6-Rse binding and/or Gas6-X, wherein X is a known receptor of Gas6.

In another embodiment, PCDGF receptor agonists of the invention activate Rse and ultimately inhibit Rse expression.

The present invention also provides methods for inhibiting PCDGF-induced Rse activity in a mammal, comprising administering to the mammal a composition that comprises an Rse-immunoglobulin or a PCDGF antagonist.

In another embodiment, PCDGF receptor antagonists of the invention prevent or inhibit the binding of PCDGF to Rse, but are not anti-angiogenic.

In another preferred embodiment, a method of enhancing the survival or proliferation of a mammalian cell, preferably, a neuron, or glial cell, such as a Schwann cell is provided by administering an effective amount of PCDGF or fragments thereof.

In another preferred embodiment, PCDGF or fragments thereof which activate Rse, are used to treat neurological diseases or disorder where PCDGF enhances the survival or proliferation of a mammalian cell, preferably, a neuron, glial cell, such as a Schwann cell.

In yet another preferred embodiment, an antitumor composition of the invention comprises an antibody or fragment thereof capable of interfering with the binding of PCDGF to Rse, but does not inhibit angiogenesis.

Preferred Antagonists of the invention

Antibodies

As discussed above, the invention encompasses administration of antibodies (preferably monoclonal antibodies) or fragments thereof that immunospecifically bind to and antagonize Rse signaling (“PCDGF antagonistic antibodies”); inhibit a cancer cell phenotype, e.g., inhibit colony formation in soft agar or tubular network formation in a three-dimensional basement membrane or extracellular matrix preparation, such as MATRIGEL™ (“cancer cell phenotype inhibiting antibodies”); preferentially bind epitopes on PCDGF; and/or bind PCDGF with a K_{off} of less than 3×10⁻³ s⁻¹.

In one embodiment, the antibody binds to the extracellular domain of Rse and, preferably, also antagonizes Rse, e.g., decreases Rse phosphorylation.

Antibodies of the invention include, but are not limited to, monoclonal antibodies, synthetic antibodies, recombinantly produced antibodies, intrabodies, multispecific antibodies, human antibodies, humanized antibodies, chimeric antibodies, single-chain Fvs (scFv), single chain antibodies, Fab fragments, F(ab′) fragments, disulfide-linked Fvs (sdFv), and epitope-binding fragments of any of the above. In particular, antibodies used in the methods of the present invention include immunoglobulin molecules and immunologically active portions of immunoglobulin molecules, i.e., molecules that contain an antigen binding site that immunospecifically binds to PCDGF or Rse and is an antagonist of PCDGF, inhibits or reduces a cancer cell phenotype, preferentially binds an PCDGF or Rse epitope exposed on cancer cells but not non-cancer cells, and/or binds PCDGF or Rse with a K_{off} of less than 3×10⁻³ s⁻¹. The immunoglobulin molecules of the invention can be of any type (e.g., IgG, IgE, IgM, IgD, IgA and IgY), class (e.g., IgG₁, IgG₂, IgG₃, IgG₄, IgA₁ and IgA₂) or subclass of immunoglobulin molecule.

The antibodies used in the methods of the invention may be from any animal origin including birds and mammals (e.g., human, murine, donkey, sheep, rabbit, goat, guinea pig, camel, horse, or chicken). Preferably, the antibodies are human or humanized monoclonal antibodies. As used herein, “human” antibodies include antibodies having the amino acid sequence of a human immunoglobulin and include antibodies isolated from human immunoglobulin libraries or from mice that express antibodies from human genes.

The antibodies used in the methods of the present invention may be monospecific, bispecific, trispecific or of greater multispecificity. Multispecific antibodies may immunospecifically bind to different epitopes of a PCDGF or Rse polypeptide or may immunospecifically bind to both an PCDGF or Rse polypeptide as well a heterologous epitope, such as a heterologous polypeptide or solid support material. See, e.g., International Publication Nos. WO 93/17715, WO 92/08802, WO 91/00360, and WO 92/05793; Tutt, et al., 1991, J. Immunol. 147:60-69; U.S. Pat. Nos. 4,474,893, 4,714,681, 4,925,648, 5,573,920, and 5,601,819; and Kostelny et al., 1992, J. Immunol. 148:1547-1553.

The antibodies used in the methods of the invention include derivatives that are modified, i.e., by the covalent attachment of any type of molecule to the antibody such that covalent attachment. For example, but not by way of limitation, the antibody derivatives include antibodies that have been modified, e.g., by glycosylation, acetylation, pegylation, phosphorylation, amidation, derivatization by known protecting/blocking groups, proteolytic cleavage, linkage to a cellular ligand or other protein, etc. Any of numerous chemical modifications may be carried out by known techniques, including, but not limited to, specific chemical cleavage, acetylation, formylation, metabolic synthesis of tunicamycin, etc. Additionally, the derivative may contain one or more non-classical amino acids.

The present invention encompasses single domain antibodies, including camelized single domain antibodies (see e.g., Muyldermans et al., 2001, Trends Biochem. Sci. 26:230; Nuttall et al., 2000, Cur. Pharm. Biotech. 1:253; Reichmann and Muyldermans, 1999, J. Immunol. Meth. 231:25; International Publication Nos. WO 94/04678 and WO 94/25591; U.S. Pat. No. 6,005,079; which are incorporated herein by reference in their entireties).

The methods of the present invention also encompass the use of antibodies or fragments thereof that have half-lives (e.g., serum half-lives) in a mammal, preferably a human, of greater than 15 days, preferably greater than 20 days, greater than 25 days, greater than 30 days, greater than 35 days, greater than 40 days, greater than 45 days, greater than 2 months, greater than 3 months, greater than 4 months, or greater than 5 months. The increased half-lives of the antibodies of the present invention or fragments thereof in a mammal, preferably a human, results in a higher serum titer of said antibodies or antibody fragments in the mammal, and thus, reduces the frequency of the administration of said antibodies or antibody fragments and/or reduces the concentration of said antibodies or antibody fragments to be administered. Antibodies or fragments thereof having increased in vivo half-lives can be generated by techniques known to those of skill in the art. For example, antibodies or fragments thereof with increased in vivo half-lives can be generated by modifying (e.g., substituting, deleting or adding) amino acid residues identified as involved in the interaction between the Fc domain and the FcRn receptor (see, e.g., International Publication No. WO 97/34631 and U.S. patent application Ser. No. 10/020,354 filed Dec. 12, 2001 entitled “Molecules With Extended Half-Lives, Compositions and Uses Thereof,” which are incorporated herein by reference in their entireties).

Antibodies or fragments thereof with increased in vivo half-lives can be generated by attaching to said antibodies or antibody fragments polymer molecules such as high molecular weight polyethyleneglycol (PEG). PEG can be attached to said antibodies or antibody fragments with or without a multifunctional linker either through site-specific conjugation of the PEG to the N- or C-terminus of said antibodies or antibody fragments or via epsilon-amino groups present on lysine residues. Linear or branched polymer derivatization that results in minimal loss of biological activity will be used. The degree of conjugation will be closely monitored by SDS-PAGE and mass spectrometry to ensure proper conjugation of PEG molecules to the antibodies. Unreacted PEG can be separated from antibody-PEG conjugates by, e.g., size exclusion or ion-exchange chromatography.

The present invention also encompasses antibodies or fragments thereof that immunospecifically bind to PCDGF and agonize PCDGF or Rse and inhibits tumor cell proliferation or growth.

The present invention also encompasses antibodies that are bispecific.

In a preferred embodiment, antibodies of the invention are bispecific T cell engagers (BiTEs). Bispecific T cell engagers (BiTE) are bispecific antibodies that can redirect T cells for antigen-specific elimination of targets. A BiTE molecule has an antigen-binding domain that binds to a T cell antigen (e.g. CD3) at one end of the molecule and an antigen binding domain that will bind to an antigen on the target cell. A BiTE molecule was recently described in WO 99/54440, which is herein incorporated by reference. This publication describes a novel single-chain multifunctional polypeptide that comprises binding sites for the CD19 and CD3 antigens (CD19×CD3). This molecule was derived from two antibodies, one that binds to CD19 on the B cell and an antibody that binds to CD3 on the T cells. The variable regions of these different antibodies are linked by a polypeptide sequence, thus creating a single molecule. Also described, is the linking of the variable heavy chain (VH) and light chain (VL) of a specific binding domain with a flexible linker to create a single chain, bispecific antibody.

In an embodiment of this invention, an antibody or ligand that immunospecifically binds to Rse will comprise a portion of the BiTE molecule. For example, the VH and/or VL (preferably a scFv) of an antibody that binds Rse can be fused to an anti-CD3 binding portion such as that of the molecule described above, thus creating a BiTE molecule that targets Rse. In addition to the variable heavy and or light chain of antibody against Rse, other molecules that bind Rse can comprise the BiTE molecule, for example PCDGF. In another embodiment, the BiTE molecule can comprise a molecule that binds to other T cell antigens (other than CD3). For example, ligands and/or antibodies that immunospecifically bind to T-cell antigens like CD2, CD4, CD8, CD11a, TCR, and CD28 are contemplated to be part of this invention. This list is not meant to be exhaustive but only to illustrate that other molecules that can immunospecifically bind to a T cell antigen can be used as part of a BiTE molecule. These molecules can include the VH and/or VL portions of the antibody or natural ligands (for example LFA3 whose natural ligand is CD3).

In another embodiment of the invention, antagonists of the invention specifically block PCDGF-induced cyclin D1 expression and/or PCDGF-induced expression and phosphorylation of MAPK.

The “binding domain” as used in accordance with the present invention denotes a domain comprising a three-dimensional structure capable of specifically binding to an epitope like native antibodies, free scFv fragments or one of their corresponding immunoglobulin chains, preferably the VH chain. Thus, said domain can comprise the VH and/or VL domain of an antibody or an immunoglobulin chain, preferably at least the VH domain or more preferably the VH and VL domain linked by a flexible polypeptide linker (scFv). On the other hand, said binding domain contained in the polypeptide of the invention may comprise at least one complementarity determining region (CDR) of an antibody or immunoglobulin chain recognizing an antigen on the T cell or a cellular antigen. In this respect, it is noted that the binding domain present in the polypeptide of the invention may not only be derived from antibodies but also from other T cell or cellular antigen binding protein, such as naturally occurring surface receptors or ligands. It is further contemplated that in an embodiment of the invention, said first and or second domain of the above-described polypeptide mimic or correspond to a VH and VL region from a natural antibody. The antibody providing the binding site for the polypeptide of the invention can be, e.g., a monoclonal antibody, polyclonal antibody, chimeric antibody, humanized antibody, bispecific antibody, synthetic antibody, antibody fragment, such as Fab, Fv or scFv fragments etc., or a chemically modified derivative of any of these.

Antibody Conjugates

The present invention encompasses the use of antibodies or fragments thereof recombinantly fused or chemically conjugated (including both covalent and non-covalent conjugations) to a heterologous polypeptide (or portion thereof, preferably to a polypeptide of at least 10, at least 20, at least 30, at least 40, at least 50, at least 60, at least 70, at least 80, at least 90 or at least 100 amino acids) to generate fusion proteins. The fusion does not necessarily need to be direct, but may occur through linker sequences. For example, antibodies may be used to target heterologous polypeptides to particular cell types, either in vitro or in vivo, by fusing or conjugating the antibodies to antibodies specific for particular cell surface receptors. Antibodies fused or conjugated to heterologous polypeptides may also be used in in vitro immunoassays and purification methods using methods known in the art. See e.g., International Publication WO 93/21232; EP 439,095; Naramura et al., 1994, Immunol. Lett. 39:91-99; U.S. Pat. No. 5,474,981; Gillies et al., 1992, PNAS 89:1428-1432; and Fell et al., 1991, J. Immunol. 146:2446-2452, which are incorporated by reference in their entireties.

The present invention further includes compositions comprising heterologous polypeptides fused or conjugated to antibody fragments. For example, the heterologous polypeptides may be fused or conjugated to a Fab fragment, Fd fragment, Fv fragment, F(ab)₂ fragment, or portion thereof. Methods for fusing or conjugating polypeptides to antibody portions are known in the art. See, e.g., U.S. Pat. Nos. 5,336,603, 5,622,929, 5,359,046, 5,349,053, 5,447,851, and 5,112,946; EP 307,434; EP 367,166; International Publication Nos. WO 96/04388 and WO 91/06570; Ashkenazi et al., 1991, PNAS 88: 10535-10539; Zheng et al., 1995, J. Immunol. 154:5590-5600; and Vil et al., 1992, PNAS 89:11337-11341 (said references incorporated by reference in their entireties).

DNA shuffling may be employed to alter the activities of antibodies of the invention or fragments thereof (e.g., antibodies or fragments thereof with higher affinities and lower dissociation rates). See, generally, U.S. Pat. Nos. 5,605,793; 5,811,238; 5,830,721; 5,834,252; and 5,837,458, and Patten et al., 1997, Curr. Opinion Biotechnol. 8:724-33; Harayama, 1998, Trends Biotechnol. 16:76; Hansson, et al., 1999, J. Mol. Biol. 287:265; and Lorenzo and Blasco, 1998, BioTechniques 24:308 (each of these patents and publications are hereby incorporated by reference in its entirety). Antibodies or fragments thereof, or the encoded antibodies or fragments thereof, may be altered by being subjected to random mutagenesis by error-prone PCR, random nucleotide insertion or other methods prior to recombination. One or more portions of a polynucleotide encoding an antibody or antibody fragment, which portions immunospecifically bind to PCDGF (or Rse) may be recombined with one or more components, motifs, sections, parts, domains, fragments, etc. of one or more heterologous molecules.

Moreover, the antibodies or fragments thereof can be fused to marker sequences, such as a peptide to facilitate purification. In preferred embodiments, the marker amino acid sequence is a hexa-histidine peptide, such as the tag provided in a pQE vector (QIAGEN, Inc., 9259 Eton Avenue, Chatsworth, Calif., 91311), among others, many of which are commercially available. As described in Gentz et al., 1989, PNAS 86:821, for instance, hexa-histidine provides for convenient purification of the fusion protein. Other peptide tags useful for purification include, but are not limited to, the hemagglutinin “HA” tag, which corresponds to an epitope derived from the influenza hemagglutinin protein (Wilson et al., 1984, Cell 37:767) and the “flag” tag.

In other embodiments, antibodies of the present invention or fragments or variants thereof conjugated to a diagnostic or detectable agent. Such antibodies can be useful for monitoring or prognosing the development or progression of a cancer as part of a clinical testing procedure, such as determining the efficacy of a particular therapy. Such diagnosis and detection can accomplished by coupling the antibody to detectable substances including, but not limited to various enzymes, such as but not limited to horseradish peroxidase, alkaline phosphatase, beta-galactosidase, or acetylcholinesterase; prosthetic groups, such as but not limited to streptavidin/biotin and avidin/biotin; fluorescent materials, such as but not limited to, umbelliferone, fluorescein, fluorescein isothiocynate, rhodamine, dichlorotriazinylamine fluorescein, dansyl chloride or phycoerythrin; luminescent materials, such as but not limited to, luminol; bioluminescent materials, such as but not limited to, luciferase, luciferin, and aequorin; radioactive materials, such as but not limited to, bismuth (²¹³Bi), carbon (¹⁴C), chromium (⁵¹Cr), cobalt (⁵⁷Co), fluorine (¹⁸F), gadolinium (¹⁵³Gd, ¹⁵⁹Gd), gallium (⁶⁸Ga, ⁶⁷Ga), germanium (⁶⁸Ge), holmium (¹⁶⁶Ho), indium (115In, ¹¹³In, ¹¹²In, ¹¹¹In), iodine (¹³¹I, ¹²⁵I, ¹²³I, ¹²¹I), lanthanium (¹⁴⁰La), lutetium (¹⁷⁷Lu), manganese (⁵⁴Mn), molybdenum (⁹⁹Mo), palladium (¹⁰³Pd), phosphorous (³²P), praseodymium (¹⁴²Pr), promethium (¹⁴⁹Pm), rhenium (¹⁸⁶Re, ¹⁸⁸Re), rhodium (¹⁰⁵Rh), ruthemium (⁹⁷Ru), samarium (¹⁵³Sm), scandium (⁴⁷Sc), selenium (⁷⁵Se), strontium (⁸⁵Sr), sulfur (³⁵S), technetium (⁹⁹Tc), thallium (²⁰¹Ti), tin (¹¹³Sn, ¹¹⁷Sn), tritium (³H), xenon (¹³³Xe), ytterbium (⁶⁹Yb, ¹⁷⁵Yb), yttrium (⁹⁰Y), zinc (⁶⁵Zn); positron emitting metals using various positron emission tomographies, and nonradioactive paramagnetic metal ions.

The present invention further encompasses uses of antibodies or fragments thereof conjugated to a therapeutic agent.

An antibody or fragment thereof may be conjugated to a therapeutic moiety such as a cytotoxin, e.g., a cytostatic or cytocidal agent, a therapeutic agent or a radioactive metal ion, e.g., alpha-emitters. A cytotoxin or cytotoxic agent includes any agent that is detrimental to cells. Examples include paclitaxel, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, puromycin, epirubicin, and cyclophosphamide and analogs or homologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BCNU) and lomustine (CCNU), cyclothosphamide, busulfan, dibromomannitol, streptozotocin, mitomycin C, and cisdichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

Further, an antibody or fragment thereof may be conjugated to a therapeutic agent or drug moiety that modifies a given biological response. Therapeutic agents or drug moieties are not to be construed as limited to classical chemical therapeutic agents. For example, the drug moiety may be a protein or polypeptide possessing a desired biological activity. Such proteins may include, for example, a toxin such as abrin, ricin A, Onconase (or another cytoxic RNase), pseudomonas exotoxin, cholera toxin, or diphtheria toxin; a protein such as tumor necrosis factor, α-interferon, β-interferon, nerve growth factor, platelet derived growth factor, tissue plasminogen activator, an apoptotic agent, e.g., TNF-α, TNF-β, AIM I (see, International Publication No. WO 97/33899), AIM II (see, International Publication No. WO 97/34911), Fas Ligand (Takahashi et al., 1994, J. Iminunol., 6:1567), and VEGI (see, International Publication No. WO 99/23105), a thrombotic agent or an anti-angiogenic agent, e.g., angiostatin or endostatin; or, a biological response modifier such as, for example, a lymphokine (e.g., interleukin-1 (“IL-1”), interleukin-2 (“IL-2”), interleukin-6 (“IL-6”), granulocyte macrophage colony stimulating factor (“GM-CSF”), and granulocyte colony stimulating factor (“G-CSF”)), or a growth factor (e.g., growth hormone (“GH”)).

Moreover, an antibody can be conjugated to therapeutic moieties such as a radioactive materials or macrocyclic chelators useful for conjugating radiometal ions (see above for examples of radioactive materials). In certain embodiments, the macrocyclic chelator is 1,4,7,10-tetraazacyclododecane-N,N′,N″,N″-tetraacetic acid (DOTA) which can be attached to the antibody via a linker molecule. Such linker molecules are commonly known in the art and described in Denardo et al., 1998, Clin Cancer Res. 4:2483-90; Peterson et al., 1999, Bioconjug. Chem. 10:553; and Zimmerman et al., 1999, Nucl. Med. Biol. 26:943-50 each incorporated by reference in their entireties.

Techniques for conjugating therapeutic moieties to antibodies are well known, see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., 1982, Immunol. Rev. 62:119-58.

Alternatively, an antibody can be conjugated to a second antibody to form an antibody heteroconjugate as described by Segal in U.S. Pat. No. 4,676,980, which is incorporated herein by reference in its entirety.

Antibodies may also be attached to solid supports, which are particularly useful for immunoassays or purification of the target antigen. Such solid supports include, but are not limited to, glass, cellulose, polyacrylamide, nylon, polystyrene, polyvinyl chloride or polypropylene.

Soluble Splice Variants of Gas 6 and Ax1 may Act as Antagonists of PCDGF

It has recently been published in the literature that soluble forms of Ax1 and Gas6 are present in the supernatant of cells as well as the serum of animals. These soluble forms can bind to each other and prevent Gas6 from binding to the fall-length active Ax1 receptor. Since PCDGF may bind Ax1 and/or its family members, e.g., Mer, the soluble forms of Ax1 and Gas6 are useful to antagonize PCDGF. This binding to PCDGF would enhance the activity of other cancer therapies by decreasing PCDGF serum levels and provide a novel mechanism for the treatment of cancer. Thus, one preferred embodiment of the invention is a method of antagonizing PCDGF using soluble fragments of Gas6 and/or Ax1 and/or Rse. Further, another preferred embodiment of the invention is a method of treating cancer, e.g., breast cancer, by administering to a patient a therapeutically effective amount of soluble fragments of Gas6 and/or Ax1 alone or in combination with other known cancer therapeutics.

Methods of Producing Antibodies

The antibodies or fragments thereof can be produced by any method known in the art for the synthesis of antibodies, in particular, by chemical synthesis or preferably, by recombinant expression techniques.

Monoclonal antibodies can be prepared using a wide variety of techniques known in the art including the use of hybridoma, recombinant, and phage display technologies, or a combination thereof. For example, monoclonal antibodies can be produced using hybridoma techniques including those known in the art and taught, for example, in Harlow et al., Antibodies: A Laboratory Manual, (Cold Spring Harbor Laboratory Press, 2nd ed. 1988); Hammerling, et al., in: Monoclonal Antibodies and T-Cell Hybridomas 563-681 (Elsevier, N.Y., 1981) (said references incorporated by reference in their entireties). The term “monoclonal antibody” as used herein is not limited to antibodies produced through hybridoma technology. The term “monoclonal antibody” refers to an antibody that is derived from a single clone, including any eukaryotic, prokaryotic, or phage clone, and not the method by which it is produced.

Methods for producing and screening for specific antibodies using hybridoma technology are routine and well known in the art. Briefly, mice can be immunized with PCDGF or Rse (either the full length protein or a domain thereof, e.g., the extracellular domain) and once an immune response is detected, e.g., antibodies specific for PCDGF are detected in the mouse serum, the mouse spleen is harvested and splenocytes isolated. The splenocytes are then fused by well known techniques to any suitable myeloma cells, for example cells from cell line SP20 available from the ATCC. Hybridomas are selected and cloned by limited dilution. Additionally, a RIMMS (repetitive immunization, multiple sites) technique can be used to immunize an animal (Kilpatrick et al., 1997, Hybridoma 16:381-9, incorporated herein by reference in its entirety). Hybridoma clones are then assayed by methods known in the art for cells that secrete antibodies capable of binding a polypeptide of the invention. Ascites fluid, which generally contains high levels of antibodies, can be generated by immunizing mice with positive hybridoma clones.

Accordingly, monoclonal antibodies can be generated by culturing a hybridoma cell secreting an antibody of the invention wherein, preferably, the hybridoma is generated by fusing splenocytes isolated from a mouse immunized with PCDGF or fragment thereof with myeloma cells and then screening the hybridomas resulting from the fusion for hybridoma clones that secrete an antibody able to bind PCDGF.

Antibody fragments, which recognize specific PCDGF or Rse epitopes, may be generated by any technique known to those of skill in the art. For example, Fab and F(ab′)2 fragments of the invention may be produced by proteolytic cleavage of immunoglobulin molecules, using enzymes such as papain (to produce Fab fragments) or pepsin (to produce F(ab′)2 fragments). F(ab′)2 fragments contain the variable region, the light chain constant region and the CH1 domain of the heavy chain. Further, the antibodies of the present invention can also be generated using various phage display methods known in the art.

In phage display methods, functional antibody domains are displayed on the surface of phage particles that carry the polynucleotide sequences encoding them. In particular, DNA sequences encoding VH and VL domains are amplified from animal cDNA libraries (e.g., human or murine cDNA libraries of lymphoid tissues). The DNA encoding the VH and VL domains are recombined together with an scFv linker by PCR and cloned into a phagemid vector (e.g., p CANTAB 6 or pComb 3 HSS). The vector is electroporated in E. coli and the E. coli is infected with helper phage. Phage used in these methods are typically filamentous phage including fd and M13 and the VH and VL domains are usually recombinantly fused to either the phage gene III or gene VIII. Phage expressing an antigen binding domain that binds to the PCDGF epitope of interest can be selected or identified with antigen, e.g., using labeled antigen or antigen bound or captured to a solid surface or bead. Examples of phage display methods that can be used to make the antibodies of the present invention include those disclosed in Brinkman et al., 1995, J. Immunol. Methods 182:41-50; Ames et al., 1995, J. Immunol. Methods 184:177; Kettleborough et al., 1994, Eur. J. Immunol. 24:952-958; Persic et al., 1997, Gene 187:9; Burton et al., 1994, Advances in Immunology 57:191-280; International Application No. PCT/GB91/01134; International Publication Nos. WO 90/02809, WO 91/10737, WO 92/01047, WO 92/18619, WO 93/11236, WO 95/15982, WO 95/20401, and WO 97/13844; and U.S. Pat. Nos. 5,698,426, 5,223,409, 5,403,484, 5,580,717, 5,427,908, 5,750,753, 5,821,047, 5,571,698, 5,427,908, 5,516,637, 5,780,225, 5,658,727, 5,733,743 and 5,969,108; each of which is incorporated herein by reference in its entirety.

As described in the above references, after phage selection, the antibody coding regions from the phage can be isolated and used to generate whole antibodies, including human antibodies, or any other desired antigen binding fragment, and expressed in any desired host, including mammalian cells, insect cells, plant cells, yeast, and bacteria, e.g., as described below. Techniques to recombinantly produce Fab, Fab′ and F(ab′)2 fragments can also be employed using methods known in the art such as those disclosed in International Publication No. WO 92/22324; Mullinax et al., 1992, BioTechniques 12:864; Sawai et al., 1995, AJRI34:26; and Better et al., 1988, Science 240:1041 (said references incorporated by reference in their entireties).

The ability of antibodies of the invention to antagonize PCDGF biological activity may be screened and measured using an assay well-known in the art. See, e.g., U.S. Pat. No. 5,955,420, which is incorporated by reference herein in its entirety.

To generate whole antibodies, PCR primers including VH or VL nucleotide sequences, a restriction site, and a flanking sequence to protect the restriction site can be used to amplify the VH or VL sequences in scFv clones. Utilizing cloning techniques known to those of skill in the art, the PCR amplified VH domains can be cloned into vectors expressing a VH constant region, e.g., the human gamma 4 constant region, and the PCR amplified VL domains can be cloned into vectors expressing a VL constant region, e.g., human kappa or lambda constant regions. Preferably, the vectors for expressing the VH or VL domains comprise an EF-1α promoter, a secretion signal, a cloning site for the variable domain, constant domains, and a selection marker such as neomycin. The VH and VL domains may also be cloned into one vector expressing the necessary constant regions. The heavy chain conversion vectors and light chain conversion vectors are then co-transfected into cell lines to generate stable or transient cell lines that express full-length antibodies, e.g., IgG, using techniques known to those of skill in the art.

For some uses, including in vivo use of antibodies in humans and in vitro detection assays, it may be preferable to use human or chimeric antibodies. Completely human antibodies are particularly desirable for therapeutic treatment of human subjects. Human antibodies can be made by a variety of methods known in the art including phage display methods described above using antibody libraries derived from human immunoglobulin sequences. See also U.S. Pat. Nos. 4,444,887 and 4,716,111; and International Publication Nos. WO 98/46645, WO 98/50433, WO 98/24893, WO 98/16654, WO 96/34096, WO 96/33735, and WO 91/10741; each of which is incorporated herein by reference in its entirety.

Human antibodies can also be produced using transgenic mice which are incapable of expressing functional endogenous immunoglobulins, but which can express human immunoglobulin genes. For example, the human heavy and light chain immunoglobulin gene complexes may be introduced randomly or by homologous recombination into mouse embryonic stem cells. Alternatively, the human variable region, constant region, and diversity region may be introduced into mouse embryonic stem cells in addition to the human heavy and light chain genes. The mouse heavy and light chain immunoglobulin genes may be rendered non-functional separately or simultaneously with the introduction of human immunoglobulin loci by homologous recombination. In particular, homozygous deletion of the J_{H} region prevents endogenous antibody production. The modified embryonic stem cells are expanded and microinjected into blastocysts to produce chimeric mice. The chimeric mice are then be bred to produce homozygous offspring that express human antibodies. The transgenic mice are immunized in the normal fashion with a selected antigen, e.g., all or a portion of a polypeptide of the invention. Monoclonal antibodies directed against the antigen can be obtained from the immunized, transgenic mice using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA, IgM and IgE antibodies. For an overview of this technology for producing human antibodies, see Lonberg and Huszar (1995, Int. Rev. Immunol. 13:65-93). For a detailed discussion of this technology for producing human antibodies and human monoclonal antibodies and protocols for producing such antibodies, see, e.g., International Publication Nos. WO 98/24893, WO 96/34096, and WO 96/33735; and U.S. Pat. Nos. 5,413,923, 5,625,126, 5,633,425, 5,569,825, 5,661,016, 5,545,806, 5,814,318, and 5,939,598, which are incorporated by reference herein in their entirety. In addition, companies such as Abgenix, Inc. (Freemont, Calif.), Genpharm (San Jose, Calif.) and Medarex (Princeton, N.J.) can be engaged to provide human antibodies directed against a selected antigen using technology similar to that described above.

A chimeric antibody is a molecule in which different portions of the antibody are derived from different immunoglobulin molecules such as antibodies having a variable region derived from a non-human antibody and a human immunoglobulin constant region. Methods for producing chimeric antibodies are known in the art. See e.g., Morrison, 1985, Science 229:1202; Oi et al., 1986, BioTechniques 4:214; Gillies et al., 1989, J. Immunol. Methods 125:191-202; and U.S. Pat. Nos. 5,807,715, 4,816,567, and 4,816,397, which are incorporated herein by reference in their entirety. Chimeric antibodies comprising one or more CDRs from a non-human species and framework regions from a human immunoglobulin molecule can be produced using a variety of techniques known in the art including, for example, CDR-grafting (EP 239,400; International Publication No. WO 91/09967; and U.S. Pat. Nos. 5,225,539, 5,530,101, and 5,585,089), veneering or resurfacing (EP 592,106; EP 519,596; Padlan, 1991, Molecular Immunology 28(4/5):489-498; Studnicka et al., 1994, Protein Engineering 7:805; and Roguska et al., 1994, PNAS 91:969), and chain shuffling (U.S. Pat. No. 5,565,332).

Polypeptides and Polynucleotides of the Invention

Polypeptides of the invention include, but are not limited to, Rse and PCDGF polypeptides, including full-length polypeptides, fragments, variants or derivatives of the full-length, known splice variants of Rse and PCDGF, and epitopes of Rse and PCDGF. Polypeptides of the invention further include, but are not limited to, polypeptide antagonists of Rse and PCDGF polypeptides. In certain embodiments of the invention, said polypeptides of the invention are at least 60%, or at least 65%, or at least 70%, or at least 75%, or at least 80%, or at least 85%, or at least 90%, or at least 95%, or at least 98%, or at least 99%, or at least 99.5% identical to the amino acid sequence of Rse and/or PCDGF.

Polypeptides of the invention further include, but are not limited to, Gas6, Mer and Ax1 polypeptides, including full-length polypeptides, fragments, variants or derivatives of the full-length, and known splice variants of Gas6, Mer and Ax1. In certain embodiments of the invention, said polypeptides of the invention are at least 60%, or at least 65%, or at least 70%, or at least 75%, or at least 80%, or at least 85%, or at least 90%, or at least 95%, or at least 98%, or at least 99%, or at least 99.5% identical to the amino acid sequence of Gas6, Mer and/or Ax1.

Polynucleotides of the invention include, but are not limited to, Rse and PCDGF polynucleotides, which encode full-length polypeptides, fragments, variants or derivatives of the full-length, known splice variants of Rse and PCDGF, and epitopes of Rse and PCDGF. Polynucleotides of the invention include, but are not limited to polynucleotides which encode polypeptide antagonists of Rse and PCDGF polypeptides. In certain embodiments of the invention, said polynucleotides of the invention are at least 60%, or at least 65%, or at least 70%, or at least 75%, or at least 80%, or at least 85%, or at least 90%, or at least 95%, or at least 98%, or at least 99%, or at least 99.5% identical to the polynucleotide sequence of Rse and/or PCDGF.

Polynucleotides of the invention include, but are not limited to, polynucleotides which encode Gas6, Mer and Ax1 polypeptides, including full-length polypeptides, fragments, variants or derivatives of the full-length, and known splice variants of Gas6, Mer and Ax1. In certain embodiments of the invention, said polynucleotides of the invention are at least 60%, or at least 65%, or at least 70%, or at least 75%, or at least 80%, or at least 85%, or at least 90%, or at least 95%, or at least 98%, or at least 99%, or at least 99.5% identical to the polynucleotide sequence of Gas6, Mer and/or Ax1.

The present invention further relates to polynucleotides which hybridize to the hereinabove-described sequences if there is at least 70%, preferably at least 90%, and more preferably at least 95% identity between the sequences. The present invention particularly relates to polynucleotides which hybridize under stringent conditions to the hereinabove-described polynucleotides.

Epitopes

The present invention encompasses polypeptides comprising, or alternatively consisting of, an epitope of the Rse and PCDGF polypeptides described in detail above or encoded by a polynucleotide that hybridizes to the complement of the sequence of Rse and PCDGF coding sequences described in detail above, under stringent hybridization conditions or lower stringency hybridization conditions as defined supra. The present invention further encompasses polynucleotide sequences encoding an epitope of a polypeptide sequence of the, polynucleotide sequences of the complementary strand of a polynucleotide sequence encoding an epitope of the invention, and polynucleotide sequences which hybridize to the complementary strand under stringent hybridization conditions or lower stringency hybridization conditions defined supra.

The term “epitopes,” as used herein, refers to portions of a polypeptide having antigenic or immunogenic activity in an animal, preferably a mammal, and most preferably in a human. In a preferred embodiment, the present invention encompasses a polypeptide comprising an epitope, as well as the polynucleotide encoding this polypeptide. An “immunogenic epitope,” as used herein, is defined as a portion of a protein that elicits an antibody response in an animal, as determined by any method known in the art, for example, by the methods for generating antibodies described infra. (See, for example, Geysen et al., Proc. Natl. Acad. Sci. USA 81:3998-4002 (1983)). The term “antigenic epitope,” as used herein, is defined as a portion of a protein to which an antibody can immunospecifically bind its antigen as determined by any method well known in the art, for example, by the immunoassays described herein. Immunospecific binding excludes non-specific binding but does not necessarily exclude cross-reactivity with other antigens. Antigenic epitopes need not necessarily be immunogenic.

Fragments that function as epitopes may be produced by any conventional means. (See, e.g., Houghten, Proc. Natl. Acad. Sci. USA 82:5131-5135 (1985), further described in U.S. Pat. No. 4,631,211).

In the present invention, antigenic epitopes preferably contain a sequence of at least 4, at least 5, at least 6, at least 7, more preferably at least 8, at least 9, at least 10, at least 15, at least 20, at least 25, and, most preferably, between about 15 to about 30 amino acids. Preferred polypeptides comprising immunogenic or antigenic epitopes are at least 10, 15, 20, 25, 30, 35, 40, 45, 50, 55, 60, 65, 70, 75, 80, 85, 90, 95, or 100 amino acid residues in length. Antigenic epitopes are useful, for example, to raise antibodies, including monoclonal antibodies, that specifically bind the epitope. Antigenic epitopes can be used as the target molecules in immunoassays. (See, for instance, Wilson et al., Cell 37:767-778 (1984); Sutcliffe et al., Science 219:660-666 (1983)).

Similarly, immunogenic epitopes can be used, for example, to induce antibodies according to methods well known in the art. (See, for instance, Sutcliffe et al., supra; Wilson et al., supra; Chow et al., Proc. Natl. Acad. Sci. USA 82:910-914; and Bittle et al., J. Gen. Virol. 66:2347-2354 (1985). The polypeptides comprising one or more immunogenic epitopes may be presented for eliciting an antibody response together with a carrier protein, such as an albumin, to an animal system (such as, for example, rabbit or mouse), or, if the polypeptide is of sufficient length (at least about 25 amino acids), the polypeptide may be presented without a carrier. However, immunogenic epitopes comprising as few as 8 to 10 amino acids have been shown to be sufficient to raise antibodies capable of binding to, at the very least, linear epitopes in a denatured polypeptide (e.g., in Western blotting).

Epitope-bearing polypeptides of the present invention may be used to induce antibodies according to methods well known in the art including, but not limited to, in vivo immunization, in vitro immunization, and phage display methods. See, e.g., Sutcliffe et al., supra; Wilson et al., supra, and Bittle et al., J. Gen. Virol., 66:2347-2354 (1985). If in vivo immunization is used, animals may be immunized with free peptide; however, anti-peptide antibody titer may be boosted by coupling the peptide to a macromolecular carrier, such as keyhole limpet hemacyanin (KLH) or tetanus toxoid. For instance, peptides containing cysteine residues may be coupled to a carrier using a linker such as maleimidobenzoyl-N-hydroxysuccinimide ester (MBS), while other peptides may be coupled to carriers using a more general linking agent such as glutaraldehyde. Animals such as, for example, rabbits, rats, and mice are immunized with either free or carrier-coupled peptides, for instance, by intraperitoneal and/or intradermal injection of emulsions containing about 100 micrograms of peptide or carrier protein and Freund's adjuvant or any other adjuvant known for stimulating an immune response. Several booster injections may be needed, for instance, at intervals of about two weeks, to provide a useful titer of anti-peptide antibody that can be detected, for example, by ELISA assay using free peptide adsorbed to a solid surface. The titer of anti-peptide antibodies in serum from an immunized animal may be increased by selection of anti-peptide antibodies, for instance, by adsorption to the peptide on a solid support and elution of the selected antibodies according to methods well known in the art.

As one of skill in the art will appreciate, and as discussed above, the polypeptides of the present invention comprising an immunogenic or antigenic epitope can be fused to other polypeptide sequences. For example, the polypeptides of the present invention may be fused with the constant domain of immunoglobulins (IgA, IgE, IgG, IgM), or portions thereof (CH1, CH2, CH3, or any combination thereof and portions thereof) resulting in chimeric polypeptides. Such fusion proteins may facilitate purification and may increase half-life in vivo. This has been shown for chimeric proteins consisting of the first two domains of the human CD4-polypeptide and various domains of the constant regions of the heavy or light chains of mammalian immunoglobulins. See, e.g., EP 394,827; Traunecker et al., Nature, 331:84-86 (1988). IgG Fusion proteins that have a disulfide-linked dimeric structure due to the IgG portion disulphide bonds have also been found to be more efficient in binding and neutralizing other molecules than monomeric polypeptides or fragments thereof alone. See, e.g., Fountoulakis et al., J. Biochem., 270:3958-3964 (1995). Nucleic acids encoding the above epitopes can also be recombined with a gene of interest as an epitope tag (e.g., the hemagglutinin (“HA”) tag or flag tag) to aid in detection and purification of the expressed polypeptide. For example, a system described by Janknecht et al. allows for the ready purification of non-denatured fusion proteins expressed in human cell lines (Janknecht et al., 1991, Proc. Natl. Acad. Sci. USA 88:8972-897). In this system, the gene of interest is subcloned into a vaccinia recombination plasmid such that the open reading frame of the gene is translationally fused to an amino-terminal tag consisting of six histidine residues. The tag serves as a matrix-binding domain for the fusion protein. Extracts from cells infected with the recombinant vaccinia virus are loaded onto Ni.sup.2+ nitriloacetic acid-agarose column and histidine-tagged proteins can be selectively eluted with imidazole-containing buffers.

Additional fusion proteins of the invention may be generated through the techniques of gene-shuffling, motif-shuffling, exon-shuffling, and/or codon-shuffling (collectively referred to as “DNA shuffling”). DNA shuffling may be employed to modulate the activities of polypeptides of the invention, such methods can be used to generate polypeptides with altered activity, as well as agonists and antagonists of the polypeptides. See, generally, U.S. Pat. Nos. 5,605,793; 5,811,238; 5,830,721; 5,834,252; and 5,837,458, and Patten et al., Curr. Opinion Biotechnol. 8:724-33 (1997); Harayama, Trends Biotechnol. 16(2):76-82 (1998); Hansson, et al., J. Mol. Biol. 287:265-76 (1999); and Lorenzo and Blasco, Biotechniques 24(2):308-13 (1998) (each of these patents and publications are hereby incorporated by reference in its entirety). In one embodiment, alteration of Rse and PCDGF polynucleotides and the polypeptides encoded by these polynucleotides may be achieved by DNA shuffling. DNA shuffling involves the assembly of two or more DNA segments by homologous or site-specific recombination to generate variation in the polynucleotide sequence. In another embodiment, polynucleotides of the invention, or the encoded polypeptides, may be altered by being subjected to random mutagenesis by error-prone PCR, random nucleotide insertion or other methods prior to recombination. In another embodiment, one or more components, motifs, sections, parts, domains, fragments, etc., of a polynucleotide coding a polypeptide of the invention may be recombined with one or more components, motifs, sections, parts, domains, fragments, etc. of one or more heterologous molecules. (each of these patents and publications are hereby incorporated by reference). In one embodiment, alteration of Rse and/or PCDGF polynucleotides and corresponding polypeptides may be achieved by DNA shuffling. DNA shuffling involves the assembly of two or more DNA segments into a desired Rse and/or PCDGF molecule by homologous, or site-specific, recombination. In another embodiment, Rse and/or PCDGF polynucleotides and corresponding polypeptides may be altered by being subjected to random mutagenesis by error-prone PCR, random nucleotide insertion or other methods prior to recombination. In another embodiment, one or more components, motifs, sections, parts, domains, fragments, etc., of Rse and/or PCDGF may be recombined with one or more components, motifs, sections, parts, domains, fragments, etc. of one or more heterologous molecules.

Preferred Therapeutic Applications

Therapeutic Targeting of PCDGF and/or its Binding Partners (e.g., Rse) on Tumor Cells in ER-Positive and ER-Negative Cancers (e.g., Breast Cancer)

Breast cancer initiation and progression is generally understood to involve changes in the expression of estrogen receptor (ERα) expression and function. Normally, the mammary gland has low levels of estrogen receptor, which regulate breast epithelial cell cells growth and involution during menstruation, pregnancy and lactation.

Early in the progression of breast cancer, ERα expression is upregulated, resulting in a histological appearance of ERα (referred to as ER-positive disease). The upregulation of ERα, when activated by ligand (β-estradiol or E2), serves to upregulate breast cancer cell growth and survival. Antagonists of ERα-E2 binding (e.g., antiestrogens such as tamoxifen, raloxifene, SERMs (see, e.g., U.S. Pat. No. 6,300,367, which is incorporated by reference herein) negatively regulate breast cancer cell growth and survival.

As the disease progresses, ERα function or expression is lost (often referred to as ER-negative disease), which has many important consequences on clinical outcome. First, loss of ERα renders these tumor cells insensitive to anti-estrogens. In addition, loss of hormone sensitivity relates to metastasis and loss of chemotherapy sensitivity. Consequently, therapeutic targeting of the causes of hormone insensitivity could have application for treatment of both ER-positive and more advanced forms of the disease.

PCDGF has been linked with ERα function in both ER-positive and ER-negative disease states. In ER-positive disease, one consequence of ER-E2 binding is upregulation of PCDGF. Thus, upregulation of ERα in breast cancer likely serves to increase PCDGF levels in cancer. In ER-negative disease, PCDGF is upregulated by mechanisms that are independent of ERα. The causes of PCDGF function are unclear but it is significant that ectopic overexpression of PCDGF in hormone-sensitive tumor cells is sufficient to facilitate tamoxifen insensitivity.

Thus, the invention encompasses therapeutic targeting of PCDGF and/or its binding partners (e.g., Rse) on tumor cells, both for ER-positive and ER-negative breast cancer. Furthermore, therapeutic targeting of these molecules could have application for treatment of other features of ER-negative disease, including metastasis and resistance to chemical and radiation therapy (beyond tamoxifen). In addition, the invention encompasses use of PCDGF and/or its binding partners for the treatment of other cancers that demonstrate metastasis or drug insensitivity.

Therefore, a preferred embodiment of the invention is a method of treating or preventing ER-positive and ER-negative breast cancer, as well as, other cancers (e.g., prostate, ovarian, lung, liver, brain, hematopoietic cell cancers, blood cell cancers) by administering to a patient a therapeutically effective amount of PCDGF or Rse antagonists. In a specific embodiment, said antagonists are antibodies against, e.g., PCDGF or Rse, which interfere with PCDGF binding to Rse. In another specific embodiment, said antagonists are antibodies are antibodies against, e.g., PCDGF or Rse, which interfere with PCDGF binding to Rse, but does not substantially prevent Gas6 binding to Rse. In yet another specific embodiment, said antagonists inhibit the expression and/or activity of the Rse receptor by interfering with the binding of PCDGF to Rse itself and/or a coreceptor and/or another tyrosine kinase receptor (e.g., Ax1 and Mer).

Additional antagonists according to these embodiments are peptides and small molecules that interfere with PCDGF and Rse activation.

Another preferred embodiment of the invention is a method of drug treating cancer drug insensitivity by administering to a patient a therapeutically effective amount of PCDGF or Rse antagonists. In a specific embodiment, said antagonists are antibodies which interfere with PCDGF binding to Rse. In another specific embodiment, said antagonists are antibodies which interfere PCDGF binding to Rse, but does not substantially prevent Gas6 binding to Rse.

Another preferred embodiment of the invention is a method of treating patients that are non-responsive to currently available therapies (e.g., cancer therapies) such as chemotherapy, radiation therapy, surgery, hormonal therapy and/or biological therapy/immunotherapy by administering to a patient a therapeutically effective amount of PCDGF or Rse antagonists.

Hyperproliferative Diseases

PCDGF is overexpressed in a number of tumor cell types (e.g., breast and ovarian cancer, lymphoma). Rse is also expressed in a number of tumor types and tumor cell lines (e.g., liver and breast cancer and leukemia). Therefore, one embodiment of the invention is to utilize a PCDGF or Rse antagonists of the invention to treat or detect hyperproliferative disorders, including neoplasms, where Rse and PCDGF are expressed.

A PCDGF or Rse polypeptide antagonists of the invention may inhibit the proliferation of the disorder through direct or indirect interactions. For instance, antagonists of the invention may inhibit the expression and/or activity of the Rse receptor by interfering with the binding of PCDGF to Rse itself and/or a coreceptor and/or another tyrosine kinase receptor (e.g., Ax1 and Mer).

Examples of hyperproliferative disorders that can be treated or detected by PCDGF or Rse antagonists of the invention include, but are not limited to, neoplasms located in the: abdomen, bone, breast, digestive system, liver, pancreas, peritoneum, endocrine glands (adrenal, parathyroid, pituitary, testicles, ovary, thymus, thyroid), eye, head and neck, nervous (central and peripheral), lymphatic system, pelvic, skin, soft tissue, spleen, thoracic, and urogenital. Examples of specific hyperproliferative disorders that can be treated or detected by PCDGF or Rse antagonists of the invention include, but are not limited to cancers of the ovary, breast, liver, prostate, kidney, testes, brain, blood cell cancers (e.g., lymphoma), hematopoietic cell cancers.

Similarly, other hyperproliferative disorders can also be treated or detected by a polynucleotide or polypeptide of the present invention. Examples of such hyperproliferative disorders include, but are not limited to: hypergammaglobulinemia, lymphoproliferative disorders, paraproteinemias, purpura, sarcoidosis, Sezary Syndrome, Waldenstron's Macroglobulinemia, Gaucher's Disease, histiocytosis, and any other hyperproliferative disease, besides neoplasia, located in an organ system listed above.

Precancerous Conditions

PCDGF is overexpressed in precancerous conditions such as PIN. Therefore, PCDGF or Rse antibodies and/or antagonists of the invention can be used to treat or detect precancerous conditions. For example, a PCDGF or Rse antagonists of the invention can be used to treat or detect DCIS, Fibrocystic disease, Cervix Dysplasia, Squamous intraepithelial lesions (SIL), Adenomatous Polyps, Barrett's esophageal dysplasia, Hepatocellular carcinoma, Adenomatous hyperplasia, Atypical adenomatous hyperplasia (AAH) of the lung, Lymphomatoid Granulomatosis (B cell), Ductal lesions, hyperplasias, or dysplasias, Prostatic intraepithelial neoplasia (PIN), Xeroderma pigmentosum, Carcinoma in situ of the skin, Actinic, or solar, Keratosis, Actinic Cheilitis, Leukoplakia, Bowen's disease, Adenomatous polyps.

Nervous System Diseases and Disorders

Schwann cells are one of the principal components of the peripheral nervous system. They play a crucial role in nerve regeneration and can be used clinically in the repair of injured nerves. It is known that Gas6, a ligand of Rse, stimulates human Schwann cell growth (see, Li et al., J. Neurosci. 1996 16(6):2012-9 and U.S. Pat. No. 5,955,420). Since it now discovered that PCDGF is a ligand for Rse, it is one object of the invention to use PCDGF polypeptides or fragments thereof, and agonists of Rse activation, to treat nervous system diseases, disorders, and/or conditions, which include, but are not limited to, nervous system injuries, and diseases, disorders, and/or conditions which result in either a disconnection of axons, a diminution or degeneration of neurons, or demyelination. Nervous system lesions which may be treated in a patient (including human and non-human mammalian patients) according to the invention, include but are not limited to, the following lesions of either the central (including spinal cord, brain) or peripheral nervous systems: (1) ischernic lesions, in which a lack of oxygen in a portion of the nervous system results in neuronal injury or death, including cerebral infarction or ischernia, or spinal cord infarction or ischemia; (2) traumatic lesions, including lesions caused by physical injury or associated with surgery, for example, lesions which sever a portion of the nervous system, or compression injuries; (3) malignant lesions, in which a portion of the nervous system is destroyed or injured by malignant tissue which is either a nervous system associated malignancy or a malignancy derived from non-nervous system tissue; (4) infectious lesions, in which a portion of the nervous system is destroyed or injured as a result of infection, for example, by an abscess or associated with infection by human immunodeficiency virus, herpes zoster, or herpes simplex virus or with Lyme disease, tuberculosis, syphilis; (5) degenerative lesions, in which a portion of the nervous system is destroyed or injured as a result of a degenerative process including but not limited to degeneration associated with Parkinson's disease, Alzheimer's disease, Huntington's chorea, or amyotrophic lateral sclerosis (ALS); (6) lesions associated with nutritional diseases, disorders, and/or conditions, in which a portion of the nervous system is destroyed or injured by a nutritional disorder or disorder of metabolism including but not limited to, vitamin B12 deficiency, folic acid deficiency, Wernicke disease, tobacco-alcohol amblyopia, Marchlafava-Bignami disease (primary degeneration of the corpus callosurn), and alcoholic cerebellar degeneration; (7) neurological lesions associated with systemic diseases including, but not limited to, diabetes (diabetic neuropathy, Bell's palsy), systemic lupus erythematosus, carcinoma, or sarcoidosis; (8) lesions caused by toxic substances including alcohol, lead, or particular neurotoxins; and (9) demyelinated lesions in which a portion of the nervous system is destroyed or injured by a demyelinating disease including, but not limited to, multiple sclerosis, human immunodeficiency virus-associated myelopathy, transverse myelopathy or various etiologies, progressive multifocal leukoencephalopathy, and central pontine myelinolysis.

In a preferred embodiment, therapeutic compositions (e.g., PCDGF polypeptide or fragments thereof) of the invention are used to protect neural cells from the damaging effects of cerebral hypoxia. According to this embodiment, the therapeutic compositions of the invention are used to treat, prevent, and/or diagnose neural cell injury associated with cerebral hypoxia. In one aspect of this embodiment, the therapeutic compositions of the invention are used to treat, prevent, and/or diagnose neural cell injury associated with cerebral ischernia. In another aspect of this embodiment, the therapeutic compositions of the invention are used to treat, prevent, and/or diagnose neural cell injury associated with cerebral infarction. In another aspect of this embodiment, the therapeutic compositions of the invention are used to treat, prevent, and/or diagnose neural cell injury associated with a stroke. In a further aspect of this embodiment, the therapeutic compositions of the invention are used to treat, prevent, and/or diagnose neural cell injury associated with a heart attack.

The compositions of the invention which are useful for treating, preventing, and/or diagnosing a nervous system disorder may be selected by testing for biological activity in promoting the survival or differentiation of neurons. For example, and not by way of limitation, therapeutic compositions of the invention which elicit any of the following effects may be useful according to the invention: (1) increased survival time of neurons in culture; (2) increased sprouting of neurons in culture or in vivo; (3) increased production of a neuron-associated molecule in culture or in vivo, e.g., choline acetyltransferase or acetylcholinesterase with respect to motor neurons; or (4) decreased symptoms of neuron dysfunction in vivo. Such effects may be measured by any method known in the art. In preferred, non-limiting embodiments, increased survival of neurons may routinely be measured using a method set forth herein or otherwise known in the art, such as, for example, the method set forth in Arakawa et al. (J. Neurosci. 10:3507-3515 (1990)); increased sprouting of neurons may be detected by methods known in the art, such as, for example, the methods set forth in Pestronk et al. (Exp. Neurol. 70:65-82 (1980)) or Brown et al. (Ann. Rev. Neurosci. 4:17-42 (1981)); increased production of neuron-associated molecules may be measured by bioassay, enzymatic assay, antibody binding, Northern blot assay, etc., using techniques known in the art and depending on the molecule to be measured; and motor neuron dysfunction may be measured by assessing the physical manifestation of motor neuron. disorder, e.g., weakness, motor neuron conduction velocity, or functional disability.

In specific embodiments, motor neuron diseases, disorders, and/or conditions that may be treated according to the invention include, but are not limited to, diseases, disorders, and/or conditions such as infarction, infection, exposure to toxin, trauma, surgical damage, degenerative disease or malignancy that may affect motor neurons as well as other components of the nervous system, as well as diseases, disorders, and/or conditions that selectively affect neurons such as amyotrophic lateral sclerosis, and including, but not limited to, progressive spinal muscular atrophy, progressive bulbar palsy, primary lateral sclerosis, infantile and juvenile muscular atrophy, progressive bulbar paralysis of childhood (Fazio-Londe syndrome), poliomyelitis and the post polio syndrome, and Hereditary Motorsensory Neuropathy (Charcot-Marie-Tooth Disease).

Nervous system diseases and disorders include, for example, central nervous system diseases, such as brain diseases (e.g., akinetic mutism, basal ganglia disease, brain abscesses, central auditory diseases (e.g., auditory perceptual disorders or central hearing loss), cerebral palsy, metabolic or chronic brain diseases, brain edemas, brain neoplasms, Canavan disease, cerebellar diseases, diffuse cerebral sclerosis, cerebrovascular diseases, dementia, encephalitis, encephalomalacia (e.g., leukomalacia), epilepsy, Hallervorden-Spatz Syndrome, hydrocephalus (e.g., Dandy-Walker Syndrome or normal pressure hydrocephalus), hypothalamic diseases (e.g., hypothalamic neoplasms), cerebral malaria, narcolepsy, cataplexy, bulbar poliomyelitis, pseudotumor cerebn', Rett Syndrome, Reye's Syndrome, thalamic diseases, cerebral toxoplasmosis, intracranial tuberculoma, or Zellweger Syndrome).

More specifically, types of basal ganglia diseases that can be treated by therapeutic compositions of the invention include, for example, drug-induced akathisia, Alzheimer's Disease, chorea, Huntington's Disease, Creutzfeldt-Jakob Syndrome, drug-induced dyskinesia, dystonia musculorum deformans, Hallervorden-Spatz Syndrome, hepatolenticular degeneration, Meige Syndrome, Neuroleptic Malignant Syndrome, Parkinson Disease (e.g., symptomatic or postencephalitic), progressive supranuclear palsy, or Tourette Syndrome.

Moreover, types of metabolic brain diseases that can be treated by therapeutic compositions of the invention, include for example, abetalipoproteinemia, gangliosidose (e.g., GMI gangliosidosis, Sandhoff Disease, or Tay-Sachs Disease), Hartnup Disease, hepatic encephalopathy, hepatolenticular degeneration, homocystinuria, kemicterus, Kinky Hair Syndrome, Leigh Disease, Lesch-Nyhan Syndrome, Maple Syrup Urine Disease, mitochondrial encephalomyopathies (e.g., MELAS Syndrome or MERRF Syndrome), central pontine myelinolysis, neuronal ceroid-lipofuscinosis, Niemann-Pick Disease, phenylketonuria, pyruvate carboxylase deficiency, pyruvate dehydrogenase complex deficiency, or Wemicke's Encephalopathy.

Additionally, types of brain neoplasms that can be treated by therapeutic compositions of the invention include, for example, cerebellar neoplasms, infratentorial neoplasms, cerebral ventricle neoplasms, choroid plexus neoplasms, hypothalamic neoplasms, or supratentorial neoplasms.

Additionally, types of central nervous system neoplasms that can be treated by therapeutic compositions of the invention include, for example, brain neoplasms (e.g., cerebellar neoplasms, infratentorial neoplasms, cerebral ventricle neoplasms, choroid plexus neoplasms, hypothalamic neoplasms, supratentorial neoplasms, meningeal neoplasms, or spinal cord neoplasms (e.g., epidural neoplasms).

Moreover, types of demyelinating diseases that can be treated by therapeutic compositions of the invention include, for example, Canavan Disease, diffuse cerebral sclerosis, adrenoleukodystrophy, encephalitis peniaxialis, globoid cell leukodystrophy, diffuse cerebral sclerosis, metachromatic leukodystrophy, allergic encephalomyelitis, necrotizing hemorrhagic encephalomyelitis, progressive multifocal leukoencephalopathy, multiple sclerosis, central pontine myelinolysis, transverse myelitis, neuromyelitis optica, scrapie, or swayback.

In further embodiments, types of encephalomyelitis that can be treated by therapeutic compositions of the invention include, for example, allergic, equine, or Venezuelan equine encephalomyelitis, necrotizing hemorrhagic encephalomyelitis, visna, or Chronic Fatigue Syndrome.

Additionally, types of spinal cord diseases that can be treated by therapeutic compositions of the invention include, for example, amyotonia congenita, amyotrophic lateral sclerosis, spinal muscular atrophy, Werdnig-Hoffinann Disease, myelitis (e.g., transverse), poliomyelitis, (e.g., bulbar and Postpollomyelitis Syndrome), spinal cord compression, spinal cord neoplasms, epidural neoplasms, syringomyelia, or tabes dorsalis.

In further embodiments, types of nervous system abnormalities that can be treated therapeutic compositions of the invention include, for example, holoprosencephaly, neural tube defects (e.g., anencephaly, hydranencephaly, amold-chiad deformity, encephalocele, meningocele, meningomyelocele, spinal dysraphism (e.g., spina bifida cystica or spina bifida occulta)), hereditary motor and sensory neuropathies (e.g., Charcot-Marie Disease, hereditary optic atrophy, Refsum's Disease, hereditary spastic paraplegia, or Werdnig-Hoffmann Disease), hereditary sensory or autonomic neuropathies (e.g., congenital analgesia or familial dysautonomia).

Additionally, types of central nervous system neoplasms that can be treated by therapeutic compositions of the invention include, for example, brain neoplasms (e.g., cerebellar neoplasms, infratentorial neoplasms, cerebral ventricle neoplasms, choroid plexus neoplasms, hypothalamic neoplasms or supratentorial neoplasms), meningeal neoplasms, spinal cord neoplasms (e.g., epidural neoplasms), peripheral nerve neoplasms (e.g., cranial nerve neoplasms, acoustic neuroma or neurofibromatosis 2).

Furthermore, nervous system diseases and disorders that can be treated by neuropeptide receptor polynucleotides or polypeptides, or agonists or antagonists of neuropeptide receptor include but are not limited to peripheral nervous system diseases such as acrodynia, amyloid neuropathies, autonomic nervous system diseases, cranial nervous system diseases, facial nerve disease, ocular motility disorders, optic nerve diseases, trigeminal neuralgia, vocal cor paralysis, demyelinating diseases, diabetic neuropathies, nerve compression syndromes, neuralgia, neuritis, hereditary motor and sensory neuropathies, hereditary sensory and autonomic neuropathies, or peripheral nerve neoplasms.

Other Preferred Indications:

The invention further encompasses therapeutic targeting of PCDGF and/or its binding partners (e.g., Rse) to treat non-cancerous diseases disease or disorders associated with increased cell growth, e.g., an autoimmune disease such as inflammatory bowel disease and psoriasis, or asthma.

In addition, the invention encompasses therapeutic targeting of PCDGF and/or its binding partners (e.g., Rse) to treat a disorder such as asthma, bronchitis, inflammatory bowel disease, emphysema, and end stage renal failure.

In addition to Rse, PCDGF may interact with other receptors for Gas6 such as Ax1 and Mer. The interaction with Gas6 and its receptors has been implicated in the pathogenesis of a number of different disease states such as nephritis, osteoporosis, rheumatoid arthritis, osteoarthritis, osteoclast bone resorption, platelet aggregation and thrombosis. Therefore, blocking PCDGF binding to Ax1 and/or Mer and/or Rse would be useful in the treatment of diseases, e.g., nephritis, osteoporosis, rheumatoid arthritis, osteoarthritis, osteoclast bone resorption, chronic allograft nephropathy, platelet aggregation, thrombosis, atherosclerosis, and/or restenosis.

Binding Activity

A polypeptide of the present invention may be used to screen for molecules that bind to the polypeptide or for molecules to which the polypeptide binds. The binding of the polypeptide and the molecule may activate (agonist), increase, inhibit (antagonist), or decrease activity of the polypeptide or the molecule bound. Examples of such molecules include antibodies, oligonucleotides, proteins (e.g., receptors), or small molecules.

Preferably, the molecule is closely related to the natural ligand of the polypeptide, e.g., a fragment of the ligand, or a natural substrate, a ligand, a structural or functional mimetic. (See, Coligan et al., Current Protocols in Immunology 1(2): Chapter 5 (1991).) Similarly, the molecule can be closely related to the natural receptor to which the polypeptide binds, or at least, a fragment of the receptor capable of being bound by the polypeptide (e.g., active site). In either case, the molecule can be rationally designed using known techniques.

Preferably, the screening for these molecules involves producing appropriate cells which express the polypeptide, either as a secreted protein or on the cell membrane. Preferred cells include cells from mammals, yeast, Drosophila, or E. coli. Cells expressing the polypeptide (or cell membrane containing the expressed polypeptide) are then preferably contacted with a test compound potentially containing the molecule to observe binding, stimulation, or inhibition of activity of either the polypeptide or the molecule.

The assay may simply test binding of a candidate compound to the polypeptide, wherein binding is detected by a label, or in an assay involving competition with a labeled competitor. Further, the assay may test whether the candidate compound results in a signal generated by binding to the polypeptide.

Alternatively, the assay can be carried out using cell-free preparations, polypeptide/molecule affixed to a solid support, chemical libraries, or natural product mixtures. The assay may also simply comprise the steps of mixing a candidate compound with a solution containing a polypeptide, measuring polypeptide/molecule activity or binding, and comparing the polypeptide/molecule activity or binding to a standard.

Preferably, an ELISA assay can measure polypeptide level or activity in a sample (e.g., biological sample) using a monoclonal or polyclonal antibody. The antibody can measure polypeptide level or activity by either binding, directly or indirectly, to the polypeptide or by competing with the polypeptide for a substrate. For example, an ELISA could be used in a competition assay where an antibody to Rse could inhibit the ability of PCDGF to bind to Rse, thereby identifying an Rse antagonist. Further, an ELISA could be used, for example, in a competition assay where an antibody to Rse could inhibit the ability of PCDGF to bind to Rse, and not inhibit the binding of Gas6 (or vice versa), thereby identifying an Rse antagonist.

PCDGF has previously been reported to induce cyclin D1 expression, as well as, induce the expression and phosphorylation of MAPK. See, e.g., U.S. Publication No. 20030099646, which is incorporated by reference herein. Therefore, an antagonists of the invention may be tested by assessing inhibition of one or both of these PCDGF-induced activities.

All of these above assays can be used as diagnostic or prognostic markers. The molecules discovered using these assays can be used to treat disease or to bring about a particular result in a patient (e.g., tumor cell death) by activating or inhibiting the polypeptide/molecule. Moreover, the assays can discover agents which may inhibit or enhance the production of the polypeptide from suitably manipulated cells or tissues.

Therefore, the invention includes a method of identifying compounds which bind to a polypeptide of the invention comprising the steps of: (a) incubating a candidate binding compound with a polypeptide of the invention; and (b) determining if binding has occurred. Moreover, the invention includes a method of identifying agonists/antagonists comprising the steps of: (a) incubating a candidate compound with a polypeptide of the invention, (b) assaying a biological activity, and (b) determining if a biological activity of the polypeptide has been altered.

Methods of Recombinantly Producing PCDGF and Rse and Methods of Measuring Activation of Rse

Methods of recombinantly producing PCDGF and Rse and isolating the same are known in the art. See, e.g., U.S. Pat. Nos. 6,001,621 and 5,955,420 (Rse); and U.S. Pat. No. 6,309,826 and Patent Publication No. US 2003/0092661 (PCDGF referred to as “GP88”). These patents and patent publications are incorporated by reference herein in their entirety.

Method of measuring activation of Rse are also well known in the art. See, e.g., U.S. Pat. Nos. 6,001,621 and 5,955,420.

Administration of Anti-PCDGF Receptor Antibody and Compositions

The antibodies of the invention may be administered alone or in combination with other types of treatments (e.g., radiation therapy, chemotherapy, hormonal therapy, immunotherapy, anti-tumor agents, antibiotics, and immunoglobulin). Generally, administration of products of a species origin or species reactivity (in the case of antibodies) that is the same species as that of the patient is preferred. Thus, in a preferred embodiment, human antibodies, fragments derivatives, analogs, or nucleic acids, are administered to a human patient for therapy or prophylaxis.

Demonstration of Therapeutic or Prophylactic Activity

The compounds or pharmaceutical compositions of the invention are preferably tested in vitro, and then in vivo for the desired therapeutic or prophylactic activity, prior to use in humans. For example, in vitro assays to demonstrate the therapeutic or prophylactic utility of a compound or pharmaceutical composition include, the effect of a compound on a cell line or a patient tissue sample. The effect of the compound or composition on the cell line and/or tissue sample can be determined utilizing techniques known to those of skill in the art including, but not limited to, rosette formation assays and cell lysis assays. In accordance with the invention, in vitro assays which can be used to determine whether administration of a specific compound is indicated, include in vitro cell culture assays in which a patient tissue sample is grown in culture, and exposed to or otherwise administered a compound, and the effect of such compound upon the tissue sample is observed.

Therapeutic and/or Prophylactic Administration and Composition

The invention provides methods of treatment, inhibition and prophylaxis by administration to a subject of an effective amount of a compound or pharmaceutical composition of the invention, preferably an antibody of the invention. In a preferred embodiment, the compound is substantially purified (e.g., substantially free from substances that limit its effect or produce undesired side effects). The subject is preferably an animal, including but not limited to animals such as cows, pigs, horses, chickens, cats, dogs, etc., and is preferably a mammal, and most preferably human.

Formulations and methods of administration that can be employed when the compound comprises a nucleic acid or an immunoglobulin are described above; additional appropriate formulations and routes of administration can be selected from among those described herein below.

Various delivery systems are known and can be used to administer a compound of the invention, e.g., encapsulation in liposomes, microparticles, microcapsules, recombinant cells capable of expressing the compound, receptor-mediated endocytosis (see, e.g., Wu and Wu, J. Biol. Chem. 262:4429-4432 (1987)), construction of a nucleic acid as part of a retroviral or other vector, etc. Methods of introduction include but are not limited to intradermal, intramuscular, intraperitoneal, intravenous, subcutaneous, intranasal, epidural, and oral routes. The compounds or compositions may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. In addition, it may be desirable to introduce the pharmaceutical compounds or compositions of the invention into the central nervous system by any suitable route, including intraventricular and intrathecal injection; intraventricular injection may be facilitated by an intraventricular catheter, for example, attached to a reservoir, such as an Ommaya reservoir. Pulmonary administration can also be employed, e.g., by use of an inhaler or nebulizer, and formulation with an aerosolizing agent.

In a specific embodiment, it may be desirable to administer the pharmaceutical compounds or compositions of the invention locally to the area in need of treatment; this may be achieved by, for example, and not by way of limitation, local infusion during surgery, topical application, e.g., in conjunction with a wound dressing after surgery, by injection, by means of a catheter, by means of a suppository, or by means of an implant, said implant being of a porous, non-porous, or gelatinous material, including membranes, such as sialastic membranes, or fibers. Preferably, when administering a protein, including an antibody, of the invention, care must be taken to use materials to which the protein does not absorb.

In another embodiment, the compound or composition can be delivered in a vesicle, in particular a liposome (see Langer, Science 249:1527-1533 (1990); Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid.)

In yet another embodiment, the compound or composition can be delivered in a controlled release system. In one embodiment, a pump may be used (see Langer, supra; Sefton, CRC Crit. Ref. Biomed. Eng. 14:201 (1987); Buchwald et al., Surgery 88:507 (1980); Saudek et al., N. Engl. J. Med. 321:574 (1989)). In another embodiment, polymeric materials can be used (see Medical Applications of Controlled Release, Langer and Wise (eds.), CRC Press, Boca Raton, Fla. (1974); Controlled Drug Bioavailability, Drug Product Design and Performance, Smolen and Ball (eds.), Wiley, New York (1984); Ranger and Peppas, J., Macromol. Sci. Rev. Macromol. Chem. 23:61 (1983); see also Levy et al., Science 228:190 (1985); During et al., Ann. Neurol. 25:351 (1989); Howard et al., J. Neurosurg. 71:105 (1989)). In yet another embodiment, a controlled release system can be placed in proximity of the therapeutic target, i.e., the brain, thus requiring only a fraction of the systemic dose (see, e.g., Goodson, in Medical Applications of Controlled Release, supra, vol. 2, pp. 115-138 (1984)).

Other controlled release systems are discussed in the review by Langer (Science 249:1527-1533 (1990)).

In a specific embodiment where the compound of the invention is a nucleic acid encoding a protein, the nucleic acid can be administered in vivo to promote expression of its encoded protein, by constructing it as part of an appropriate nucleic acid expression vector and administering it so that it becomes intracellular, e.g., by use of a retroviral vector (see U.S. Pat. No. 4,980,286), or by direct injection, or by use of microparticle bombardment (e.g., a gene gun; Biolistic, Dupont), or coating with lipids or cell-surface receptors or transfecting agents, or by administering it in linkage to a homeobox-like peptide which is known to enter the nucleus (see e.g., Joliot et al., Proc. Natl. Acad. Sci. USA 88:1864-1868 (1991)), etc. Alternatively, a nucleic acid can be introduced intracellularly and incorporated within host cell DNA for expression, by homologous recombination.

The present invention also provides pharmaceutical compositions. Such compositions comprise a therapeutically effective amount of a compound, and a pharmaceutically acceptable carrier. In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a therapeutically effective amount of the compound, preferably in purified form, together with a suitable amount of carrier so as to provide the form for proper administration to the patient. The formulation should suit the mode of administration.

In a preferred embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The compounds of the invention can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

The amount of the compound of the invention which will be effective in the treatment, inhibition and prevention of a disease or disorder associated with aberrant expression and/or activity of a polypeptide of the invention can be determined by standard clinical techniques. In addition, in vitro assays may optionally be employed to help identify optimal dosage ranges. The precise dose to be employed in the formulation will also depend on the route of administration, and the seriousness of the disease or disorder, and should be decided according to the judgment of the practitioner and each patient's circumstances. Effective doses may be extrapolated from dose-response curves derived from in vitro or animal model test systems.

For antibodies, the dosage administered to a patient is typically 0.1 mg/kg to 100 mg/kg of the patient's body weight. Preferably, the dosage administered to a patient is between 0.1 mg/kg and 20 mg/kg of the patient's body weight, more preferably 1 mg/kg to 10 mg/kg of the patient's body weight. Generally, human antibodies have a longer half-life within the human body than antibodies from other species due to the immune response to the foreign polypeptides. Thus, lower dosages of human antibodies and less frequent administration is often possible. Further, the dosage and frequency of administration of antibodies of the invention may be reduced by enhancing uptake and tissue penetration (e.g., into the brain) of the antibodies by modifications such as, for example, lipidation.

The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects approval by the agency of manufacture, use or sale for human administration.

Diagnosis and Imaging

Labeled antibodies, and derivatives and analogs thereof, which specifically bind to a polypeptide of interest can be used for diagnostic purposes to detect, diagnose, or monitor diseases and/or disorders associated with the aberrant expression and/or activity of a polypeptide of the invention. The invention provides for the detection of aberrant expression of a polypeptide of interest, comprising (a) assaying the expression of the polypeptide of interest in cells or body fluid of an individual using one or more antibodies specific to the polypeptide interest and (b) comparing the level of gene expression with a standard gene expression level, whereby an increase or decrease in the assayed polypeptide gene expression level compared to the standard expression level is indicative of aberrant expression.

The invention provides a diagnostic assay for diagnosing a disorder, comprising (a) assaying the expression of the polypeptide of interest in cells or body fluid of an individual using one or more antibodies specific to the polypeptide interest and (b) comparing the level of gene expression with a standard gene expression level, whereby an increase or decrease in the assayed polypeptide gene expression level compared to the standard expression level is indicative of a particular disorder. With respect to cancer, the presence of a relatively high amount of transcript in biopsied tissue from an individual may indicate a predisposition for the development of the disease, or may provide a means for detecting the disease prior to the appearance of actual clinical symptoms. A more definitive diagnosis of this type may allow health professionals to employ preventative measures or aggressive treatment earlier thereby preventing the development or further progression of the cancer.

Antibodies of the invention can be used to assay protein levels in a biological sample using classical immunohistological methods known to those of skill in the art (e.g., see Jalkanen, et al., J. Cell. Biol. 101:976-985 (1985); Jalkanen, et al., J. Cell. Biol. 105:3087-3096 (1987)). Other antibody-based methods useful for detecting protein gene expression include immunoassays, such as the enzyme linked immunosorbent assay (ELISA) and the radioimmunoassay (RIA). Suitable antibody assay labels are known in the art and include enzyme labels, such as, glucose oxidase; radioisotopes, such as iodine (¹³¹I, ¹²⁵I, ¹²³I, ¹²¹I), carbon (¹⁴C), sulfur (³⁵S), tritium (³H), indium (^{115m}In, ^{113m}In, ¹¹²In, ¹¹¹In), technetium (⁹⁹Tc, ^{99m}Tc), thallium (²⁰¹Ti), gallium (⁶⁸Ga, ⁶⁷Ga), palladium (¹⁰³Pd), molybdenum (⁹⁹Mo), xenon (¹³³Xe), fluorine (¹⁸F), ¹⁵³Sm, ¹⁷⁷Lu, ¹⁵⁹Gd, ¹⁴⁹Pm, ¹⁴⁰La, ¹⁷⁵Yb, ¹⁶⁶Ho, ⁹⁰Y, ⁴⁷Sc, ¹⁸⁶Re, ¹⁸⁸Re, ¹⁴²Pr, ¹⁰⁵Rh, ⁹⁷Ru; luminol; and fluorescent labels, such as fluorescein and rhodamine, and biotin.

Techniques known in the art may be applied to label antibodies of the invention. Such techniques include, but are not limited to, the use of bifunctional conjugating agents (see e.g., U.S. Pat. Nos. 5,756,065; 5, 714,631; 5,696,239; 5,652,361; 5,505,931; 5,489,425; 5,435,990; 5,428,139; 5,342,604; 5,274,119; 4,994,560; and 5,808,003; the contents of each of which are hereby incorporated by reference in its entirety).

One embodiment of the invention is the detection and diagnosis of a disease or disorder associated with aberrant expression of a polypeptide of interest in an animal, preferably a mammal and most preferably a human. In one embodiment, diagnosis comprises: (a) administering (for example, parenterally, subcutaneously, or intraperitoneally) to a subject an effective amount of a labeled molecule which specifically binds to the polypeptide of interest; (b) waiting for a time interval following the administering for permitting the labeled molecule to preferentially concentrate at sites in the subject where the polypeptide is expressed (and for unbound labeled molecule to be cleared to background level); (c) determining background level; and (d) detecting the labeled molecule in the subject, such that detection of labeled molecule above the background level indicates that the subject has a particular disease or disorder associated with aberrant expression of the polypeptide of interest. Background level can be determined by various methods including, comparing the amount of labeled molecule detected to a standard value previously determined for a particular system. As described herein, specific embodiments of the invention are directed to the use of the antibodies of the invention to quantitate or qualitate concentrations of cells of B cell lineage or cells of monocytic lineage.

It will be understood in the art that the size of the subject and the imaging system used will determine the quantity of imaging moiety needed to produce diagnostic images. In the case of a radioisotope moiety, for a human subject, the quantity of radioactivity injected will normally range from about 5 to 20 millicuries of ^{99m}Tc. The labeled antibody or antibody fragment will then preferentially accumulate at the location of cells which contain the specific protein. In vivo tumor imaging is described in S. W. Burchiel et al., “Imnmunopharmacokinetics of Radiolabeled Antibodies and Their Fragments.” (Chapter 13 in Tumor Imaging: The Radiochemical Detection of Cancer, S. W. Burchiel and B. A. Rhodes, eds., Masson Publishing Inc. (1982).

Depending on several variables, including the type of label used and the mode of administration, the time interval following the administration for permitting the labeled molecule to preferentially concentrate at sites in the subject and for unbound labeled molecule to be cleared to background level is 6 to 48 hours or 6 to 24 hours or 6 to 12 hours. In another embodiment the time interval following administration is 5 to 20 days or 5 to 10 days.

In an embodiment, monitoring of the disease or disorder is carried out by repeating the method for diagnosing the disease or disease, for example, one month after initial diagnosis, six months after initial diagnosis, one year after initial diagnosis, etc.

Presence of the labeled molecule can be detected in the patient using methods known in the art for in vivo scanning. These methods depend upon the type of label used. Skilled artisans will be able to determine the appropriate method for detecting a particular label. Methods and devices that may be used in the diagnostic methods of the invention include, but are not limited to, computed tomography (CT), whole body scan such as position emission tomography (PET), magnetic resonance imaging (MRI), and sonography.

In a specific embodiment, the molecule is labeled with a radioisotope and is detected in the patient using a radiation responsive surgical instrument (Thurston et al., U.S. Pat. No. 5,441,050). In another embodiment, the molecule is labeled with a fluorescent compound and is detected in the patient using a fluorescence responsive scanning instrument. In another embodiment, the molecule is labeled with a positron emitting metal and is detected in the patent using positron emission-tomography. In yet another embodiment, the molecule is labeled with a paramagnetic label and is detected in a patient using magnetic resonance imaging (MRI).

1. An antitumor composition comprising an antibody or antibody fragment capable of interfering with the binding of PCDGF to Rse.

2. The composition according to claim 1, wherein said antibody or antibody fragment does not interfere with Gas6 binding to Rse.

3. An antitumor composition comprising an antibody or antibody fragment capable of interfering with the binding of PCDGF to Rse, wherein the antibody or fragment thereof does not inhibit angiogenesis.

4. A method of treating patients that are non-responsive to currently available therapies (e.g., cancer therapies) such as chemotherapy, radiation therapy, surgery, hormonal therapy and/or biological therapy/immunotherapy by administering to a patient a therapeutically effective amount of the composition of claim 1.

5. A method of treating or preventing ER-positive and ER-negative breast cancer by administering to a patient a therapeutically effective amount of the composition of claim 1.

6. A method of treating cancer drug insensitivity by administering to a patient a therapeutically effective amount of a PCDGF or Rse antagonist.

7. The composition according to claim 1, wherein said antibody or antibody fragment interfere or inhibits with the biological activity of PCDGF.

8. The composition according to claim 1, wherein said antibody or antibody fragment reduces the proliferation of tumorigenic cells by at least about 20% in vitro.

9. An isolated antagonist of Rse or PCDGF that inhibits the interaction between Rse and PCDGF.

10. The antagonist of claim 9 that is an antibody, a peptide, or a small molecule or an antibody fragment.

11. The antagonist of claim 9 that binds to Rse.

12. The antagonist of claim 9 that binds to PCDGF.

13. An antitumor composition comprising an antibody or antibody fragment capable of inhibiting the biological activity of the Rse receptor.

14. A method of treating lymphoma by administering to a patient a therapeutically effective amount of the composition of claim 1.

15. A method of treating lymphoma by administering to a patient a therapeutically effective amount of the composition of claim 13.

16. A method of treating or preventing ER-positive and ER-negative breast cancer by administering to a patient a therapeutically effective amount of soluble fragments of Ax1 and/or Gas6.


SEQUENCE LISTING (2 sequences)

SEQ ID NO: 1 - DNA, 2673 bp, Homo sapiens
atggcgctgaggcggagcatggggcggccggggctcccgccgctgccgctgccgccgccaccgcggctcgggctgctgctggcggctctggcttctctgctgctcccggagtccgccgccgcaggtctgaagctcatgggagccccggtgaagctgacagtgtctcaggggcagccggtgaagctcaactgcagtgtggaggggatggaggagcctgacatccagtgggtgaaggatggggctgtggtccagaacttggaccagttgtacatcccagtcagcgagcagcactggatcggcttcctcagcctgaagtcagtggagcgctctgacgccggccggtactggtgccaggtggaggatgggggtgaaaccgagatctcccagccagtgtggctcacggtagaaggtgtgccatttttcacagtggagccaaaagatctggcagtgccacccaatgcccctttccaactgtcttgtgaggctgtgggtccccctgaacctgttaccattgtctggtggagaggaactacgaagatcgggggacccgctccctctccatctgttttaaatgtaacaggggtgacccagagcaccatgttttcctgtgaagctcacaacctaaaaggcctggcctcttctcgcacagccactgttcaccttcaagcactgcctgcagcccccttcaacatcaccgtgacaaagctttccagcagcaacgctagtgtggcctggatgccaggtgccgatggccgagctctgctacagtcctgtacagttcaggtgacacaggccccaggaggctgggaagtcctggctgttgtggtccctgtgcccccctttacctgcctgctccgggacctggtgcctgccaccaactacagcctcagggtgcgctgtgccaatgccttggggccctctccctatgctgactgggtgccctttcagaccaagggtctagccccagccagcgctccccaaaacctccatgccatccgcacagattcaggcctcatcttggagtgggaagaagtgatccccgaggcccctttggaaggccccctgggaccctacaaactgtcctgggttcaagacaatggaacccaggatgagctgacagtggaggggaccagggccaatttgacaggctgggatccccaaaaggacctgatcgtacgtgtgtgcgtctccaatgcagttggctgtggaccctggagtcagccactggtggtctcttctcatgaccgtgcaggccagcagggccctcctcacagccgcacatcctgggtacctgtggtccttggtgtgctaacggccctggtgacggctgctgccctggccctcatcctgcttcgaaagagacggaaagagacgcggtttgggcaagcctttgacagtgtcatggcccggggagagccagccgttcacttccgggcagcccggtccttcaatcgagaaaggcccgagcgcatcgaggccacattggacagcttgggcatcagcgatgaactaaaggaaaaactggaggatgtgctcatcccagagcagcagttcaccctgggccggatgttgggcaaaggagagtttggttcagtgcgggaggcccagctgaagcaagaggatggctcctttgtgaaagtggctgtgaagatgctgaaagctgacatcattgcctcaagcgacattgaagagttcctcagggaagcagcttgcatgaaggagtttgaccatccacacgtggccaaacttgttggggtaagcctccggagcagggctaaaggccgtctccccatccccatggtcatcttgcccttcatgaagcatggggacctgcatgccttcctgctcgcctcccggattggggagaacccctttaacctacccctccagaccctgatccggttcatggtggacattgcctgcggcatggagtacctgagctctcggaacttcatccaccgagacctggctgctcggaattgcatgctggcagaggacatgacagtgtgtgtggctgacttcggactctcccggaagatctacagtggggactactatcgtcaaggctgtgcctccaaactgcctgtcaagtggctggccctggagagcctggccgacaacctgtatactgtgcagagtgacgtgtgggcgttcggggtgaccatgtgggagatcatgacacgtgggcagacgccatatgctggcatcgaaaacgctgagatttacaactacctcattggcgggaaccgcctgaaacagcctccggagtgtatggaggacgtgtatgatctcatgtaccagtgctggagtgctgaccccaagcagcgcccgagctttacttgtctgcgaatggaactggagaacatcttgggccagctgtctgtgctatctgccagccaggaccccttatacatcaacatcgagagagctgaggagcccactgcgggaggcagcctggagctacctggcagggatcagccctacagtggggctggggatggcagtggcatgggggcagtgggtggcactcccagtgactgtcggtacatactcacccccggagggctggctgagcagccagggcaggcagagcaccagccagagagtcccctcaatgagacacagaggcttttgctgctgcagcaagggctactgccacacagtagctgttag

SEQ ID NO: 2 - protein, 890 aa, Homo sapiens
MALRRSMGRPGLPPLPLPPPPRLGLLLAALASLLLPESAAAGLKLMGAPVKLTVSQGQPVKLNCSVEGMEEPDIQWVKDGAVVQNLDQLYIPVSEQHWIGFLSLKSVERSDAGRYWCQVEDGGETEISQPVWLTVEGVPFFTVEPKDLAVPPNAPFQLSCEAVGPPEPVTIVWWRGTTKIGGPAPSPSVLNVTGVTQSTMFSCEAHNLKGLASSRTATVHLQALPAAPFNITVTKLSSSNASVAWMPGADGRALLQSCTVQVTQAPGGWEVLAVVVPVPPFTCLLRDLVPATNYSLRVRCANALGPSPYADWVPFQTKGLAPASAPQNLHAIRTDSGLILEWEEVIPEAPLEGPLGPYKLSWVQDNGTQDELTVEGTRANLTGWDPQKDLIVRVCVSNAVGCGPWSQPLVVSSHDRAGQQGPPHSRTSWVPVVLGVLTALVTAAALALILLRKRRKETRFGQAFDSVMARGEPAVHFRAARSFNRERPERIEATLDSLGISDELKEKLEDVLIPEQQFTLGRMLGKGEFGSVREAQLKQEDGSFVKVAVKMLKADIIASSDIEEFLREAACMKEFDHPHVAKLVGVSLRSRAKGRLPIPMVILPFMKHGDLHAFLLASRIGENPFNLPLQTLIRFMVDIACGMEYLSSRNFIHRDLAARNCMLAEDMTVCVADFGLSRKIYSGDYYRQGCASKLPVKWLALESLADNLYTVQSDVWAFGVTMWEIMTRGQTPYAGIENAEIYNYLIGGNRLKQPPECMEDVYDLMYQCWSADPKQRPSFTCLRMELENILGQLSVLSASQDPLYINIERAEEPTAGGSLELPGRDQPYSGAGDGSGMGAVGGTPSDCRYILTPGGLAEQPGQAEHQPESPLNETQRLLLLQQGLLPHSSC